(19) United States
(12) Patent Application Publication
(10) Pub. No.: US 2013/0245545 A1
(43) Pub. Date: Sep. 19, 2013

(54) Drug Injection Devices, Systems and Methods

(71) Applicants: Ofer Arnold, Alon Ha'galil, Ha'movil (IL); Ofer Yodfat, Modi'in (IL); Oliver Shergold, Bolligen (CH)

(72) Inventors: Ofer Arnold, Alon Ha'galil, Ha'movil (IL); Ofer Yodfat, Modi'in (IL); Oliver Shergold, Bolligen (CH)

(73) Assignee: MEDINGO LTD., Yoqneam Illit (IL)

(21) Appl. No.: 13/617,403

(22) Filed: Sep. 14, 2012

(30) Foreign Application Priority Data
Sep. 20, 2011 (EP) 11007643.7

(51) Int. Cl. A61M 5/172

(52) U.S. Cl. CPC A61M 5/1723
USPC 604/66

(57) ABSTRACT

Disclosed is a drug injection device, comprising a drug delivery module and an analyte measurement module, such as a blood glucose meter. The injection device may include a support for receiving a reservoir or a cartridge, a drive means for expelling the fluid from the reservoir or cartridge, and an injection means fluidically coupled to the reservoir or cartridge. Additionally, the injection device comprises an analyte measurement module. Medical systems including embodiments of the injection device are also disclosed.

### CROSS REFERENCE TO RELATED APPLICATIONS

The present U.S. utility patent application is related to and claims the priority benefit to European Patent Application Serial No. 11007643.7, filed Sep. 20, 2011, the contents of which are hereby incorporated by reference in their entirety into this disclosure.

### BACKGROUND

The present disclosure relates generally to drug insulin injection devices. Specifically, there are described insulin pen devices integrated with a blood glucose meter adapted to diabetes management. Associated systems and methods are described.

A standard or conventional insulin pen is used to inject insulin for the treatment of diabetes. It is composed of an insulin cartridge (integrated or bought separately), a spring drive to force insulin from the cartridge and a dial to set the dose delivered. The pen is used with disposable needles. A durable pen uses a replaceable insulin cartridge. When the insulin cartridge is empty, the empty cartridge is disposed of and a new one is inserted in the pen. A prefilled pen is entirely disposable. The pen comes pre-filled with insulin, and when the insulin cartridge or reservoir is empty, the entire unit is discarded.

### SUMMARY

The present disclosure comprises devices, systems and methods of delivering a fluid.

There is disclosed an embodiment of an injection device for delivering a fluid, the injection device comprising a support for receiving a reservoir or a cartridge, a drive means for expelling the fluid from the reservoir or cartridge, an injection means fluidically coupled to the reservoir or cartridge, wherein the injection device comprises an analyte measurement module.

In at least one embodiment, the delivery of the fluid corresponds to a prick, or is configured to oscillate back and forth along a longitudinal axis as the liquid is dispensed from the fluid injection means.

In at least one embodiment, the analyte measurement module is part of a housing of the injection device.

In at least one embodiment, the analyte measurement module is a blood glucose meter and the fluid is insulin.

In at least one embodiment, the device comprises a user interface including data inputting and outputting means, such as a microphone, a speaker, a touch-sensitive screen, a slidable cursor, a physical button, a camera, or a combination thereof.

In at least one embodiment, the device further comprises communication means, adapted to communicate with other networked devices, such as an insulin pump, an insulin pen, a continuous monitoring sensor, a personal computer, a mobile phone, a tablet, a television or a combination thereof.

In at least one embodiment, the device further comprises an energy source, wherein the energy source is one of a dynamo, or a spring, or a rechargeable battery, or a solar cell or a combination thereof.

In at least one embodiment, the device comprises a code reader adapted to identify a previously coded drug cartridge.

In at least one embodiment, the device comprises a plurality of drug cartridges, prefilled or not, and/or drug reservoirs, such as rapid and long acting insulin.

In at least one embodiment, the device comprises an injection needle. The needle may in at least one embodiment be operably coupled to the injection means.

In at least one embodiment, the needle is a sprinkler needle comprising a plurality of holes.

In at least one embodiment, there is disclosed an adapter to the needle, the adapter being connectable via a tubing to an infusion set or a patch-pump inlet.

In at least one embodiment, the device comprises a cap covering the needle, containing lancing elements and one or more blood test strips compatible with the blood glucose meter.

In at least one embodiment, the device is adapted to be received in a base station, wherein the base station is suitable for energy transfer and/or data exchange.

At least one embodiment of the present disclosure is a medical system comprising an embodiment of an injection device of the present disclosure having a pen shape and comprising computing means adapted to implement one or more functions of a bolus calculator, the implementation of the computing means being local, for example in a processor, or the computing means being accessed remotely via a network.

At least one embodiment of the present disclosure is a medical system comprising a plurality of cooperating pen-shaped medical devices according to the present disclosure and/or with a standard pen mounted with a communication cap comprising a sensor and communications means.

At least one embodiment of the present disclosure is a cap for an insulin pen comprising test strips, desiccant elements, and lancing elements.

### BRIEF DESCRIPTION OF THE DRAWINGS

The features and advantages of the present disclosure, and the manner of attaining them, will be more apparent and better understood by reference to the following descriptions taken in conjunction with the accompanying figures, wherein:

FIG. 1 shows a global environment of the insulin pen or injection device interacting with other networked medical devices and IT (information technology) devices, for example, a personal computer, a mobile phone, Tablet PC, and another insulin pen, according to at least one embodiment of the present disclosure;

FIG. 2 shows an insulin pen (injection device) integrated with a BGM, according to at least one embodiment of the present disclosure;

FIG. 3 shows an injection device (insulin pen) provided with a communication channel, for example a USB port, according to at least one embodiment of the present disclosure;

FIG. 4 illustrates an exemplary workflow of the bolus calculator, according to at least one embodiment of the present disclosure;

FIG. 5 shows insulin cartridges of different sizes that can be used in an embodiment of the disclosed device, according to at least one embodiment of the present disclosure;

FIG. 6 shows an insulin pen (injection device) according to the disclosures that is provided with a bar code reader and a communication channel, according to at least one embodiment of the present disclosure;

FIGS. 7A and 7B show another exemplary insulin pen (injection device) according to the disclosure comprising a socket for receiving an insulin cartridge, according to at least one embodiment of the present disclosure;

FIG. 8 shows an embodiment of insulin pen (injection device) comprising two reservoirs, for example, comprising rapid and long acting insulin, according to at least one embodiment of the present disclosure;

FIGS. 9A to 9D show an embodiment of an adapter and method for refilling an insulin pen (injection device), according to at least one embodiment of the present disclosure;

FIG. 10 shows an exemplary user interface of an insulin pen (injection device), for example, comprising a slidable element and/or a touchscreen and/or a microphone and/or physical input button and/or a combination thereof, according to at least one embodiment of the present disclosure;

FIGS. 11A to 11G illustrates the actuation of a BGM of an embodiment of the disclosed device, according to at least one embodiment of the present disclosure;

FIG. 12 shows a particular embodiment where an insulin pen (injection device) is provided with a sprinkler needle instead of a standard needle, according to at least one embodiment of the present disclosure;

FIG. 13 shows an embodiment of an insulin pen (injection device) provided with an adapter for a connection to standard infusion set through tubing, according to at least one embodiment of the present disclosure;

FIG. 14 shows an example of an insulin pen (injection device) provided with an adapter for a direct connection to standard infusion set, according to at least one embodiment of the present disclosure;

FIGS. 15 and 16 show an indicator for a disclosed insulin pen (injection device), according to at least one embodiment of the present disclosure;

FIG. 17 shows an embodiment of a cap for an insulin pen (injection device) comprising lancing elements held within the cap of the insulin pen, according to at least one embodiment of the present disclosure;

FIG. 18 shows a base station for receiving the insulin pen (injection device) and having dual functions in this particular embodiment (recharge battery and data transfer), according to at least one embodiment of the present disclosure;

FIG. 19A shows an exemplary adapter for enabling a communication channel to a standard insulin pen, according to at least one embodiment of the present disclosure;

FIG. 19B shows an insulin pen (injection device) according to the present disclosure communicating with a standard or conventional insulin pen, according to at least one embodiment of the present disclosure;

FIG. 20 shows a motorized insulin pen (injection device) according to the disclosure, according to at least one embodiment of the present disclosure;

FIGS. 21A to 21C show the cap comprising lancing elements and test strips, as well as a strip compartment cover with desiccant, according to at least one embodiment of the present disclosure;

FIG. 22 shows steps of an example of the use of the injection device with the cap comprising lancing elements and test strips, according to at least one embodiment of the present disclosure; and

FIG. 23 shows different adaptors to accommodate different insulin cartridges, according to at least one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

Values such as amounts of insulin displayed that are discussed and/or illustrated in the figures of the present disclosure are only examples. These values may cause injuries or death if inappropriately applied.

The injection device described in the present disclosure may, in at least one embodiment, be provided with a BGM (Blood Glucose Meter, or Measurement or Monitoring system). Further, the insulin pen comprising a BGM may be hereafter referred to as the “pen-creas” insulin pen, or “intelligent” insulin pen, or as the pen according to embodiments of the present disclosure, as opposed to a standard or conventional or classic insulin pen.

A Blood Glucose Meter (or Measurement, or Monitoring) is used for testing the concentration of glucose in the blood to assess the glycemic state of a patient. A blood glucose test is performed by piercing the skin (typically on the finger) to draw blood, then applying the blood to a chemically active disposable ‘test-strip’. Persons with Type 2 diabetes test at least once per day. Persons with Type 1 usually test their blood sugar more often (3 to 10 times per day), both to assess the effectiveness of their prior insulin dose and to help determine their next insulin dose. The term “Monitoring” often refers to regular (as opposed to punctual) measurements. Both embodiments are covered in the present disclosure.

In at least one embodiment of an injection device of the present disclosure, the injection device comprises a support for receiving a reservoir or a cartridge, a drive means for expelling the fluid from the reservoir or cartridge, an injection means fluidically coupled to the reservoir wherein the injection device comprises an analyte measurement module.

Further, in an exemplary embodiment, the injection device is provided with a single housing that accommodates:
a drug delivery module, including (at least) a motor, gears, a plunger and a cartridge/reservoir;
an analyte monitoring module including (at least) a BGM;
a processor to manage the delivery of the drug, analyte sensing/monitoring, and interface (I/O) with the user;

The single housing optionally includes input means and output means. In one embodiment, the input means and output means are both implemented, at least in part, in a single touch-sensitive screen. Thus, the operation of the injection device (both drug delivery and analyte sensing/monitoring) is being done digitally at the injection device housing and is being controlled by a single processor. In some embodiments, the injection device includes a bolus calculator feature (operated in conjunction with the processor). In some embodiments, the drug/therapeutic fluid comprises insulin and the analyte comprises glucose (i.e., body glucose).

In at least one embodiment, the drug injection device presents the shape of a pen (elongated device, i.e. approximately cylindrical with a ratio length/diameter superior to 1). But in other embodiments, the shape can be very different (front view or section as a square, star, oval, triangle, rectangular, trapezoidal, polygon). The housing containing the different components can be rigid or flexible (adapted to deformations or to conform to the body of the patient). Noticeably the device can be “distributed” over several (communicating) parts and disposed on or in or near the body of the patient (system of devices).

An example of usage of an embodiment of an injection device of the present disclosure may include the following steps:
insertion of a blood glucose (BG) strip to a dedicated port located at the injection device housing;
receiving by the processor (accommodated within the housing) the BG reading/value from the strip;
inputting by a user an estimation of carbs to be ingested (for example via a touch-sensitive screen located at the housing);
determining by the processor (via bolus calculator feature) the recommended bolus amount to be delivered to the user, based on at least the BG reading and the carbs estimation. In some embodiments, other parameters (either input by the user or retrieved from a memory accommodated within the housing or elsewhere through, for example, a wireless connection) are taken into account in this bolus amount calculation. Additional parameters may include one or more of Carbs-to-Insulin Ration (“CIR”; which may be also be referred to as and insulin-to-carbs ration, or “ITC”), Insulin Sensitivity Factor (“ISF”; which may be also referred to simply as “IS”), Target Blood Glucose (“TBG”), Bolus-on-Board (“BOB”; may be also referred-to as “Insulin-On-Board” and “Residual Insulin”), and the like;
the recommended bolus amount may be presented to the user, for example, via the touch-sensitive screen;

Finally, the bolus amount is delivered (controlled by the processor). In some embodiments, the delivery may be carried out automatically (i.e., based on the bolus calculator determination without any interference of the user. In some embodiments, the delivery may be carried out “semi-automatically” with some interference of the user, for example, the user has to confirm the delivery or may change the recommended bolus amount at his/her discretion. In some embodiments, the user may reject or ignore the bolus calculator recommendation and to either deliver the drug or not.

The network to which the exemplary injection device can be connected may, for example, be any kind of network, such as Internet, Intranet, Wi-Fi, Bluetooth or a mesh network (ad-hoc network). It can correspond in particular to the network enabling a system of networked medical devices.

Effects or advantages or objectives of embodiments of the present disclosure may relate to an increased comfort of use, a better glycemic control, an optimized diabetes management, an enhanced portability, an increased discreteness, a new user interaction model, and new or complimentary administration schemes.

The disclosure enables diabetes management system in one device. The advising system is based on data collected from multiple sources (internal and external inputs). The operation can be fully automatic and electronic, including alerts, alarms, and reminders. The presented solution is compatible with off-the-shelf (all insulin vendors) and proprietary (multi-volume) cartridges.

New or complementarity treatments in diabetes management are enabled. The device indeed combines advantages and functions of different devices. For example, the presented insulin pen complements or replaces a traditional or micro or patch insulin pump (for example, during the day when the patient is awakened, with an adapted basal replacement if required), it complements or replaces a lancing device used to perform blood samples withdrawals, and it complements or replaces a stand-alone blood glucose meter.

The convenience of the use of a device in a pen shape enables increased discreteness (due to the shape factor which is similar to a widely distributed object, the attention of third parties is not drawn to the pen).

A way of using the device and the associated diabetes management can also be deeply modified. For example, it becomes possible to compensate for a basal delivery during a disconnection of an insulin pump by applying frequent injections. During sport or exercising, it is possible to administer a different (for example, a degraded) basal rate, in absence of a pump.

The portability of the device opens many new possibilities. For example, it enables a better calibration of a Continuous Monitoring sensor or device, if in use. Therefore, it enables a diabetic to make more frequent measurements in order to reach a better glycemic control.

As an intelligent and connected device, the insulin pen can cooperate with other networked medical devices and optimize the management of diabetes. For example, the bolus calculator of the “intelligent” (connected) insulin pen can manage downstream boluses versus upstream boluses (subtracting, fractioning, etc). In other words, the management of boli is improved or optimized, because of the cooperation with networked devices in an integrated system.

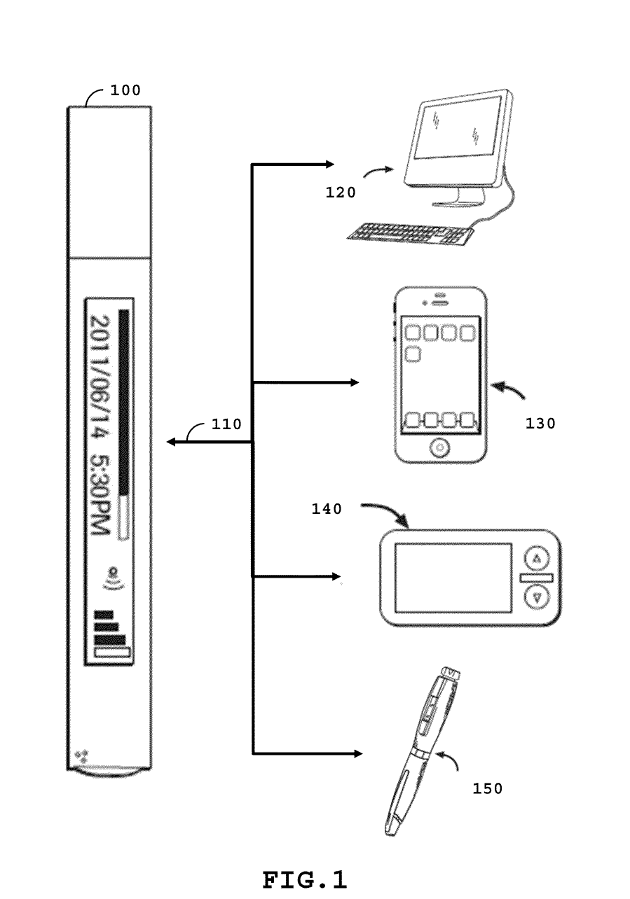

FIG. 1 illustrates an embodiment of an injection device (pen or insulin injection device) of the present disclosure capable of communicating with external devices, either medical devices such as blood glucose monitor/meter (stand alone, IBGM, CGM), dedicated remote control, insulin delivery device (e.g., pump, another pen) and/or other generic devices such as PC, laptop, PDA (dedicated or generic), cellular phone, smartphone, media player (e.g., iPod), TV, and the like. Communication may be configured as RF communication, Bluetooth, IR, and any other means of wireless communication. In some embodiments, the communication may be wired (e.g., USB cable). Communication maybe one or more of: unidirectional (e.g., injection device to external device, external device to injection device), bi-directional (e.g., injection device to and from external device) and poly-directional (e.g., injection device to and from multiple devices, communication with a network, cloud, etc.).

FIG. 1 shows an embodiment of the insulin injection device 100 in interaction through the network 110 with personal computer 120 and/or a mobile phone 130 and/or a tablet PC 140 and/or another insulin injection device (standard insulin injection device or an injection device according to one or more embodiments of the present disclosure). The insulin injection device communicates with networked medical devices and/or IT (information technology) devices. The cooperation of devices enables new interactivity schemes in diabetes management. The display of the insulin injection device 100 can be complemented by a display of the personal computer 120, when passing by, for example. One or more of the medical or IT device also can send commands to the insulin injection device 100. For example, the smartphone 140 can trigger the request for a bolus to the insulin injection device 100. A doctor can retrieve or update data via the network. All parameters of the insulin injection device, including firmware, operating system and software applications can be modified through the network in at least one embodiment of the present disclosure. Access rights or credentials can also be managed by a central logic (centralized or distributed over surrounding devices).

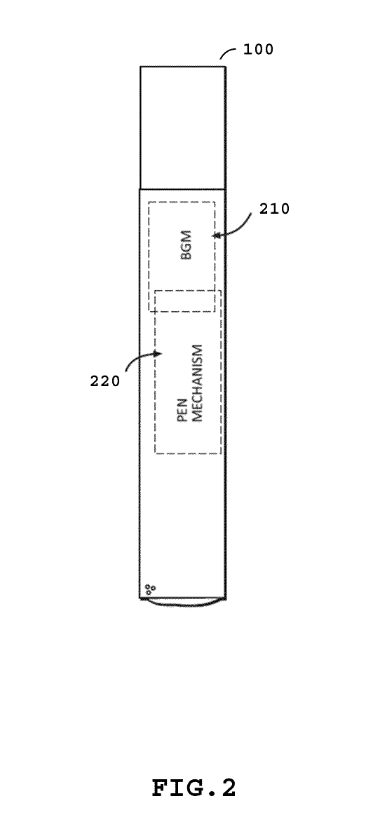

FIG. 2 illustrates an example of an injection device 100 configured as an insulin injection device including both insulin delivery means 220 (i.e., “pen/injection device mechanism”) and analyte sensing means 210, i.e., blood glucose monitoring (“BGM”).

Further, FIG. 2 shows a feature of an exemplary embodiment of an insulin injection device according to the disclosure, in that it integrates a blood glucose meter. The techniques used for measuring the blood glucose levels may be optical or electro-chemical.

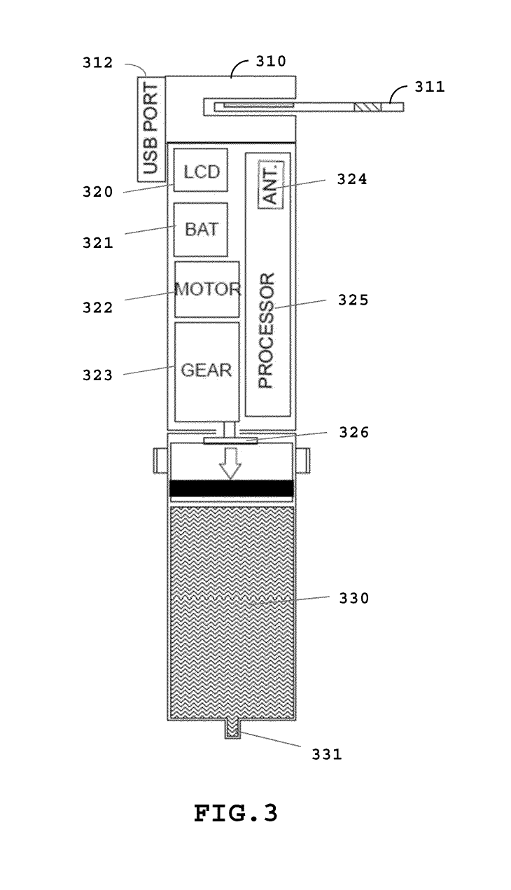

FIG. 3 provides a general description of exemplary components of an embodiment of an insulin pen or injection device. In this instance, the embodiment of the insulin pen or injection device comprises:
a reservoir 330 for retaining a drug (e.g., insulin). The reservoir can be flexible or rigid. It can be pre-filled or not. It can be disposable or reusable. The reservoir can consist in an extension connectable to the injection device or can consist in a pre-connected part. The reservoir is suitable for insulin storage, but can also be suitable for storing any other drug (for example glucagon).
a driving mechanism including a motor 322 (e.g., stepper motor, DC motor, piezoelectric motor or any other type of driving mechanism), one or more gears 323 (e.g., planetary gear), and a plunger/piston 326 for displacing the drug from the reservoir to the body of the patient. The driving mechanism can also be constituted by micro mechanisms, such as MEMS (micro-electro-mechanical systems). According to such a micrometric design, a volumetric membrane comes with a pair of check valves integrated in a MEMS chip. The chip is a stack of several layers bonded together: a Silicon-On-Insulator SOI plate with micro-machined pump structures and two Pyrex cover plates with through holes. The MEMS chip is assembled with a piezoelectric actuator that moves the membrane in reciprocating movement to compress and decompress the fluid in the pumping chamber.
a power source 321 (e.g., one or more batteries, one or more capacitors, or a micro fuel cell) for providing power at least to the driving mechanism. The battery can be rechargeable or replaceable/disposable. According to another embodiment, the source of energy comes from a dynamo and/or a capacitor for storing the energy. According to yet another embodiment, the source of energy comes from energized spring(s).
a processor/controller 325 (“CPU”). The CPU 325 can be one physical entity, but it can designate a unit which controls and/or retrieves and/or distributes computing tasks to other electronic components or via or to the local or remote network (such as cloud computing). The element 325 also designate memory means.
communication means 324 (e.g., antenna, transceiver) may communicate (wirelessly) data and/or commands with networked medical/IT devices. In one embodiment, the communication means replaces the processor 325 (where computation of the data is not performed locally). In other embodiments the communication means 324 is part of the processor/controller unit 325.
data transferring means 312 (e.g., USB port, memory card slot) for transferring data (e.g., logbooks including for example data related to infusion and/or data related to BG monitoring) to and from the injection device. The injection device can optionally comprise such ports.
input means (not shown) for entering data to the injection device and controlling at least the injection device such as one or more of: a touch-sensitive screen, key(s), keyboard, button(s), audio commands, camera, etc.
output means 320 to notify the user and provide indications (e.g., visual, audible, vibrational) such as one or more of: a touch-sensitive screen, microphone, buzzer, vibrator, pico projector, braille screen, etc.
analyte monitoring port 310 such as a port for receiving a BG (blood glucose) strip 311. The exemplary embodiment of the presently shown insulin injection device comprises a BGM (blood glucose meter).

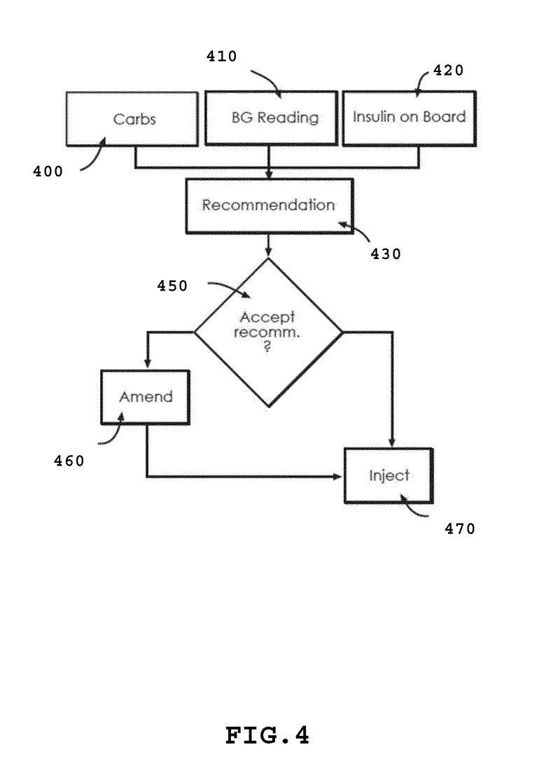

FIG. 4 shows the general usability of an embodiment of an injection device according to the present disclosure, and in particular an associated method of using the injection device. At step 400, a value of carbohydrates is received. At step 410, a blood glucose value is received from the BGM of the insulin injection device. With these two values, the embedded bolus calculator calculates a bolus recommendation 430. From this recommendation, the patient either accepts the recommendation (step 450) and injects (step 470) the bolus or amends (step 460) and injects (step 470) the bolus.

Referring to steps 410 to 420, the carbs estimation may be received via the input means (e.g., touch-sensitive screen) and may be associated with a database stored in a memory (e.g., food database, images of foods, caloric tables) and/or with a bolus calculator. The BG value(s) may be inputted via the input means, received from a remote sensor and/or received from a BG meter such as the one coupled to the BG strip port located on the injection device. The Insulin-On-Board may be inputted via input means, calculated by the processor or retrieved from a memory (step 420). Referring to step 430, the determination of the bolus amount may be carried out in various ways (local glycemic model or accessed remote model, or doctor advice for example). Referring to step 450, presenting at least the bolus amount can be implemented by the output means (e.g., touch-sensitive screen). Referring to step 460, the amendment can be implemented via input means (either at the injection device or at an external device being in communication with the injection device). Referring to step 450, if the user selects “Accept,” then the injection device may deliver the recommended bolus amount (either immediately or with a delay configurable by the user). If the user selects “Edit,” then the user may change the recommended bolus amount and only then would the injection device deliver this amount accordingly. If the user selects “Reject,” then the process of delivery is cancelled.

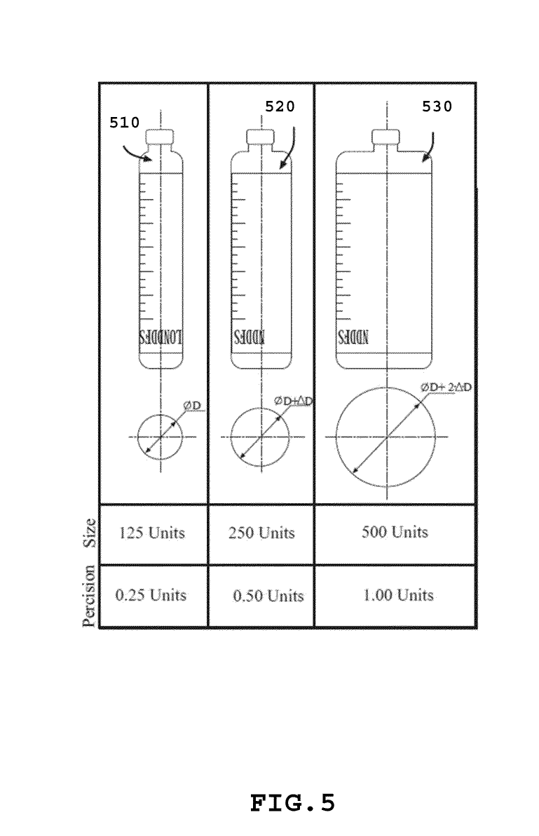

FIG. 5 illustrates various sizes of exemplary reservoirs or cartridges. Each of the cartridges is characterized by different dimensions (e.g., radius). The injection device is configured to receive the different cartridges (retaining different amount of drug). In at least one embodiment, cartridges include the identical distal ends (including the cartridge outlet) and identical proximal ends (including the interface with the plunger rod. In the first exemplary cartridge 510, the size is 125 units (precision 0.25 unit). In the second exemplary cartridge 520, the size is 250 units (precision 0.5 unit). In the third exemplary cartridge 530, the size is 500 units (precision 1 unit).

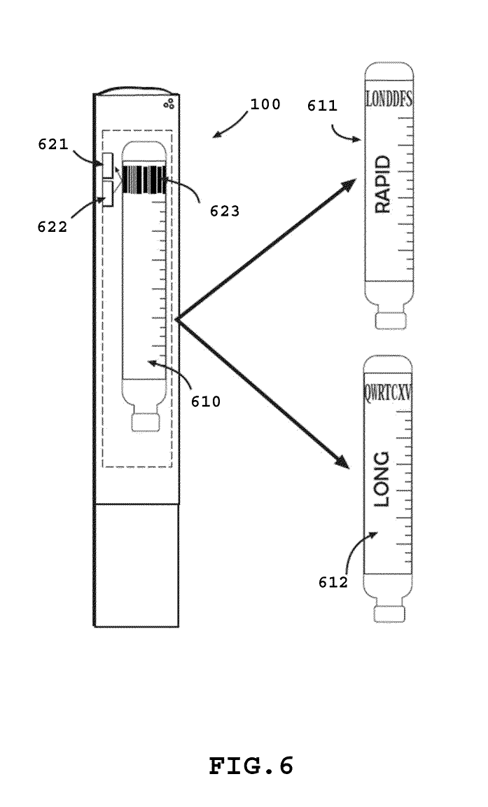

FIG. 6 illustrates an implementation of a recognition/identification mechanism, which may enables the identification of a cartridge and its characterization, for example, the type of insulin (rapid or long; different types of rapid (e.g., Lispro, Aspart) retained within the cartridge, insulin expiry date, manufacturer, cartridge batch/lot, etc.). The identification mechanism may include for example a barcode. In such an embodiment, the injection device includes a barcode reader 621/622 and the cartridge includes a corresponding barcode ID 623. In case where a non-valid cartridge is inserted into the injection device, the processor may reject it and not allow its usage.

Other identification mechanisms may include color detection, RFID, holograms, OCR, etc. Encryption and authentication mechanisms can be added.

An advantage of at least one embodiment of the present disclosure is that the consumption of insulin can be monitored (possibly in real-time). For example, insulin manufacturers can aggregate data from insulin injection devices through the network and adjust production or financial forecasts accordingly. Statistical analysis can provide useful data (detection of leaks, abnormal low or high consumptions, singular events for example), which in turn can lead to valuable remediation (alerts by SMS or messaging systems, firmware updates, etc.). Patients may additionally benefit downstream from automatic repurchasing options (when the reservoir level falls under a predefined threshold, an order for a new cartridge is prepared and/or sent). Compatibilities between insulin cartridges and insulin injection devices can be managed (the behavior of the insulin injection device can be driven by the detected cartridge type for example). Administration and dosage also can be defined according to the cartridge ID. Authentication options also can be implemented (ID codes can guarantee the quality of the drug), as well as other parameters (tracking options such as temperature history, validity date). The payment (in totality or in part) can occur at the first insertion for example, according to a particular business model. Further, reporting options may be enabled by this “intelligent” identification.

In yet another embodiment, the software implemented for the management of the disease is defined by the identification of the drug being inserted (for example, if an U500 insulin cartridge is detected, the treatment will be different compared to a U100 insulin type). Injection algorithms, bolus calculation, glycemic model, bolus patterns, as well as the user interactivity model can be modified as well.

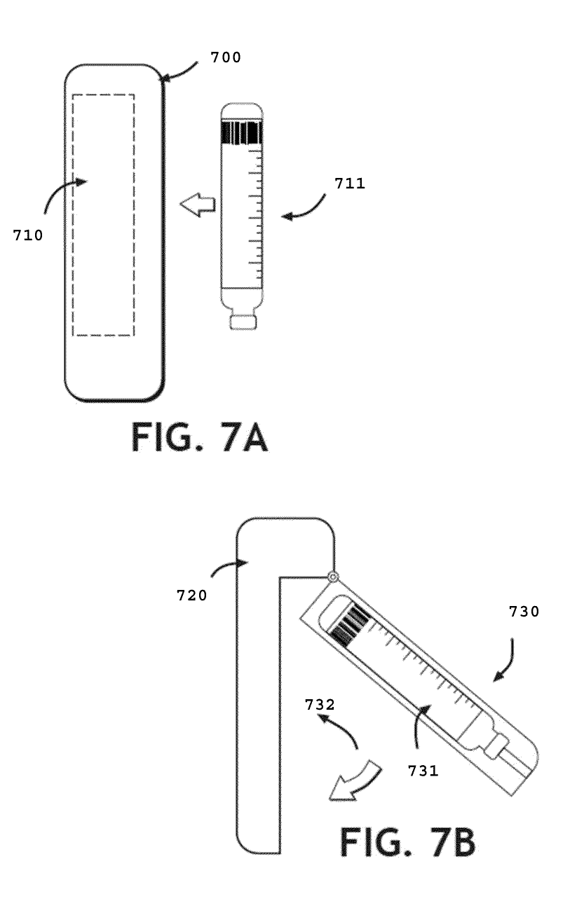

FIGS. 7A and 7B illustrate various configurations of the insertion/loading of a cartridge into the exemplary injection device. For example, as shown in FIG. 7A, the cartridge 711 may be loaded into a lateral opening/window 710 of the injection device 700. FIG. 7B shows another embodiment in which the cartridge 731 may be loaded into a hinged “barrel” 730 of injection device 720. In another embodiment (not shown), the cartridge can be loaded from a bottom opening of the injection device housing. Unlike conventional loading opening (e.g., lateral), the bottom opening enables a smaller dimension of the device (no need for an additional lateral opening, additional cover for this lateral opening), and reduction of manufacturing cost (COGS).

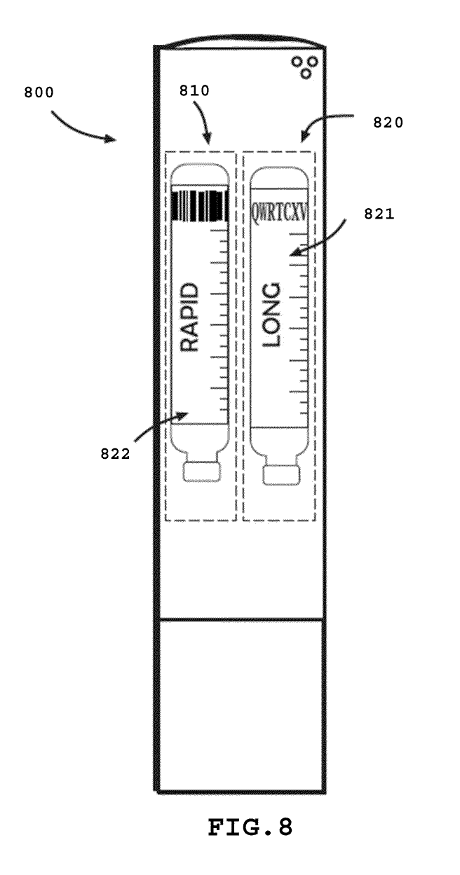

FIG. 8 shows a dual-cartridge embodiment. The injection device 800 can include a plurality of cartridges, for example, two cartridges where one cartridge retains long acting insulin 821 and another cartridge retains rapid acting insulin 822. In such an embodiment, a plurality of barcode readers 810 and 820 can be used.

The management of diabetes can leverage the presence of these two types of insulin (a bolus for a food intake with rapid insulin and basal injections with long acting insulin for example). Much more complicated schemes can be implemented as well, in order to adapt to the physiological response of the patient to the injections. In particular, the existence of a continuous blood monitoring implemented on the patient can enable an optimized glycemic control (feedback loops, real-time adjustments, machine learning algorithms, flexible rapid/long injection patterns, individualized values, etc.).

As a generalization, an embodiment of the drug injection device can comprise a plurality of cartridges (2 or more), corresponding to different drugs. Further, patient treatments can be optimized and individualized (mixing rapid and long acting insulin for example). Additionally, several diseases can be treated at the same time.

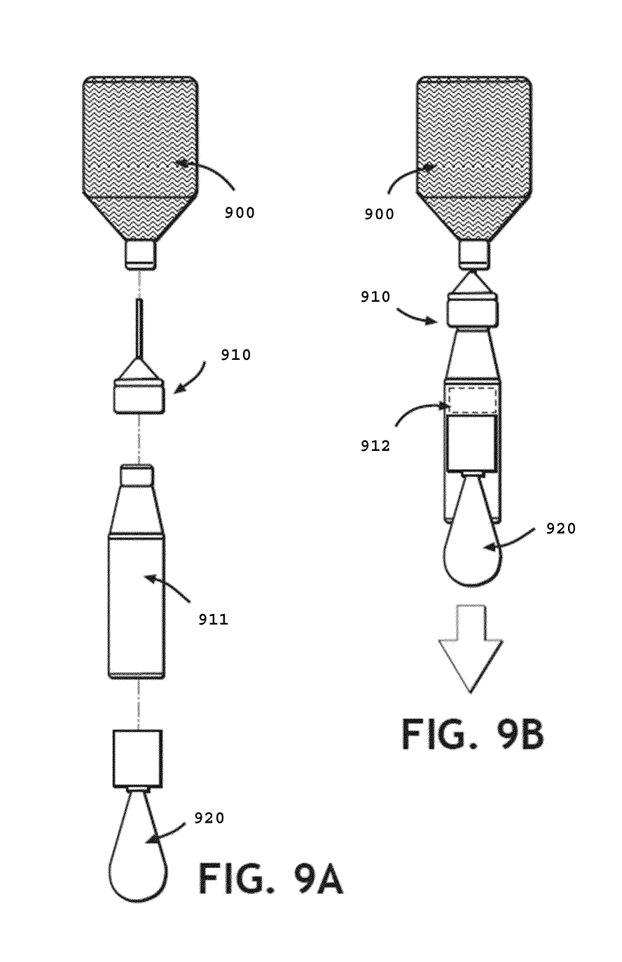

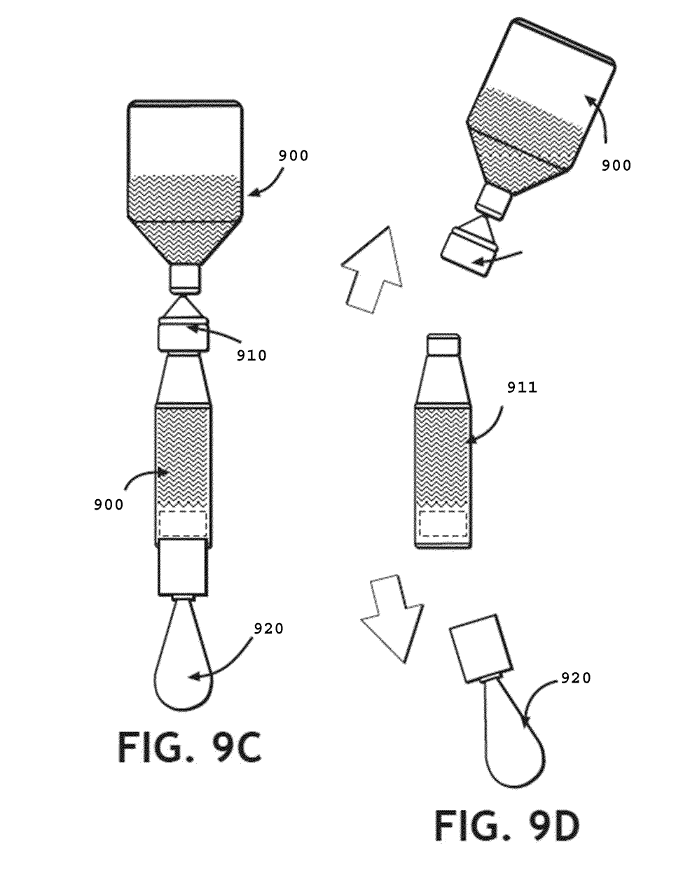

FIGS. 9A to 9D illustrate to the filling process of a cartridge 911. Such a cartridge may be formed of plastic (e.g., polypropylene) or glass and can be disposable or reusable. To transfer the drug from a vial 900 to the cartridge 911, an adapter 910 (which can be disposable) may be used. The adapter couples to the vial on the adapter's distal end and to the cartridge on the adapter's proximal end. Then a handle 920 (which can be, for example, reusable) may be coupled to the cartridge (at the cartridges proximal end). Thus, a user may fill the cartridge at his/her discretion based on their personal use and experience—one user may fill in X units of insulin and another user may choose to fill in Y units of insulin (see FIG. 9c). After filling the desired amount, the adapter and/or handle may be disposed. In another embodiment, a pre-filled cartridge may be used and loaded into the injection device.

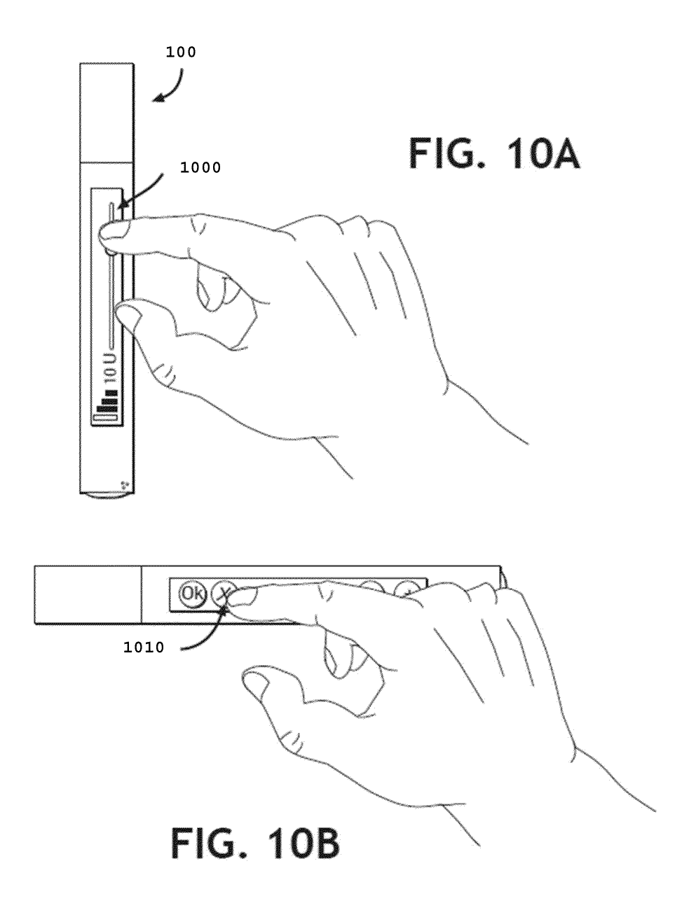

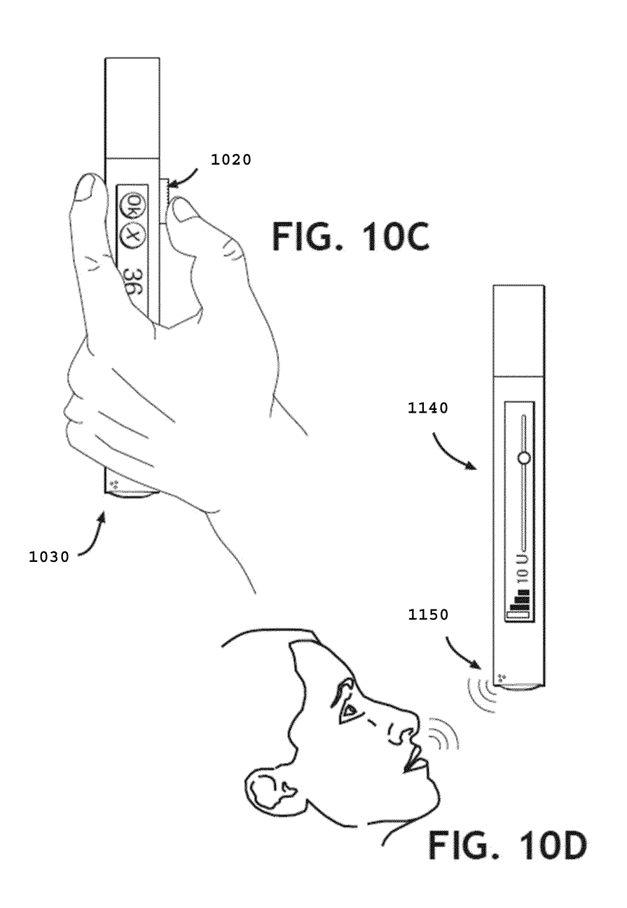

FIGS. 10A and 10B illustrate various input means located on the housing of the injection device 100. Exemplary input means may include a touch sensitive screen and/or key(s). Some embodiments may include a touch-sensitive screen on a first side of the housing of the injection device and a dedicated button on a second side, such as a bolus button. A slidable cursor 1000 (physical embodiment as a slider button such as a resistive slider or software embodiment with the touch sensitive surface) enables the user to select the desired amount of drug to be injected. User interface option 1010 enables the user to confirm, modify or cancel the bolus. The use of a slidable cursor to set the dose to be delivered by the injection device may improves discreteness, and combined with display and confirmation of the set bolus, may improve safety by ensuring that the amount injected is what is intended by the user.

FIGS. 10C and 10D illustrate the implementation of voice recognition means on the injection device to input commands/data. Embodiments of the injection device can include a microphone 1030 for receiving voice instructions, which may assist impaired/disabled patients. In at least one embodiment, the patient presses a button 1020 which triggers the activation of the microphone 1030. In another embodiment, an audio threshold level is predefined (audio signals above the threshold trigger the voice recognition system). In other embodiments, the injection device can include a speaker 1050 for audible indications. This type of indication may assist for example, visually impaired patients. In yet other embodiments, the insulin injection device or injection device also includes a camera adapted to interpret gestures, as input means. In other embodiments, a voice synthesizer can repeat what was provided by the user to ensure that the correct input has been received by the device.

Embodiments of the injection device can also be configured to enable the user to control the delivery profile (“normal” bolus, but also bolus according to an adjusted profile including a plurality of delivery rates (e.g., equivalent to a “Duo” bolus pattern).

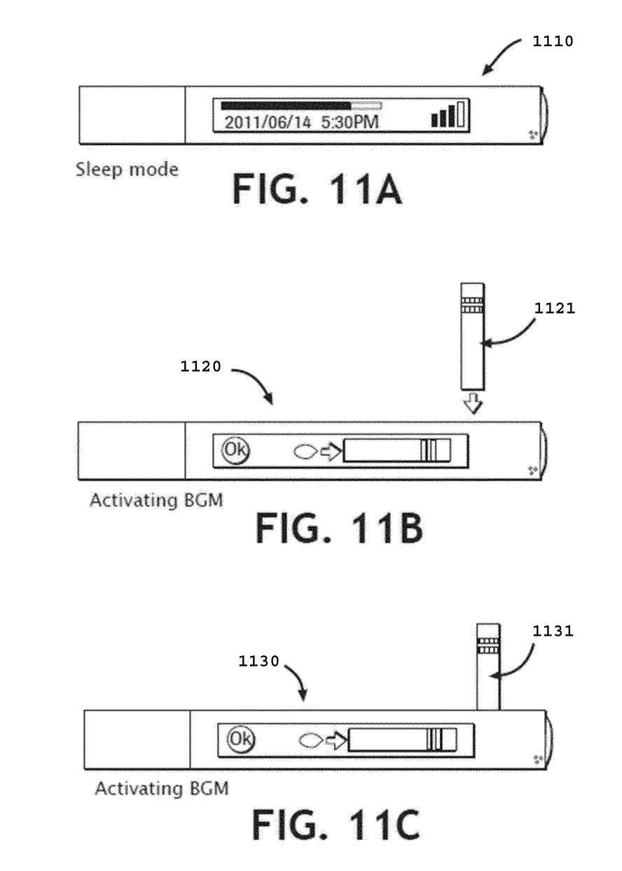

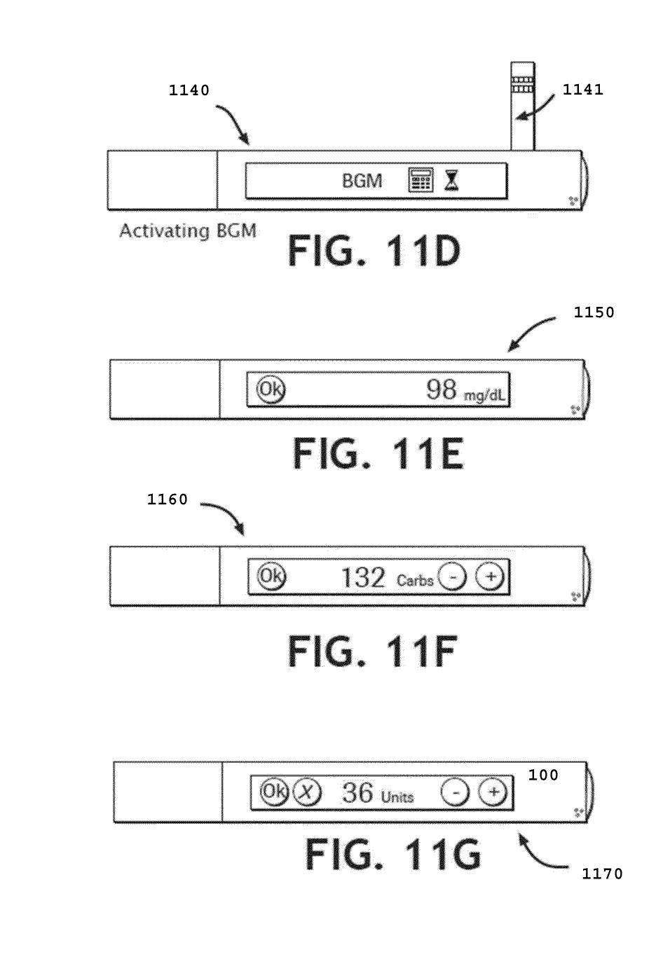

FIGS. 11A to 11G show at least one exemplary use of the disclosed insulin injection device.

When not in use, the device 1110 may operate in a sleep mode for conserving energy (FIG. 11a). The device is activated, for example, by a dedicated button/switch (or a predefined action performed at the touch-sensitive screen, by a remotely controlled command, and/or by inserting a strip into the BGM port (FIGS. 11B-C). Then, the BGM reading can be processed (FIG. 11D), the BG reading may be presented to the user (FIG. 11E), carbs estimation may be inputted or estimated and then presented (FIG. 11F), and lastly, the bolus amount recommendation can be presented to the user (FIG. 11G).

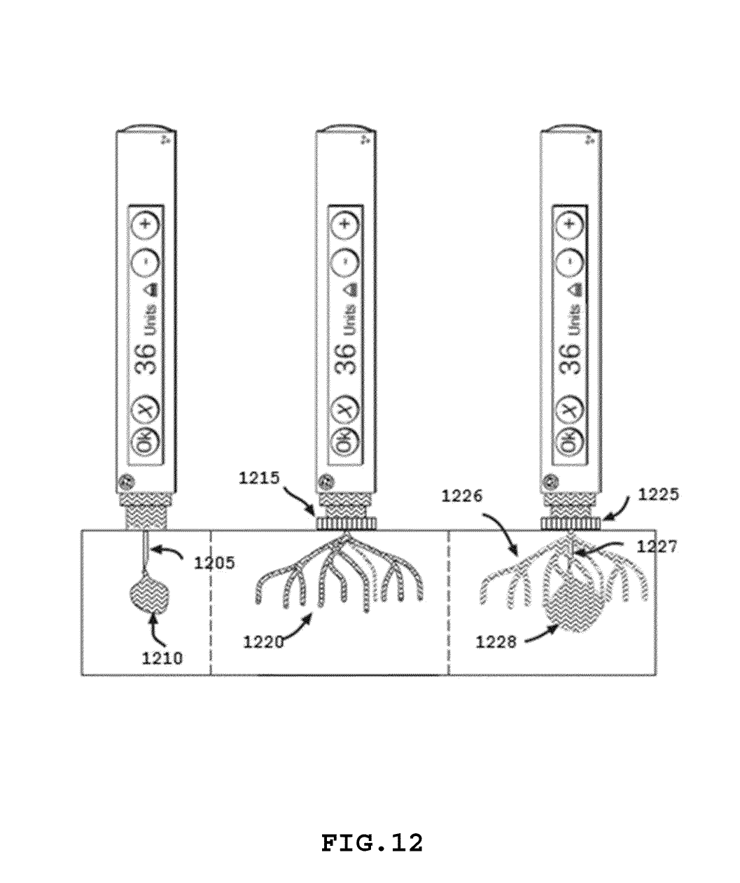

FIG. 12 illustrates an injection device with an injection means. In some embodiments, the injection means is configured as an injection needle 1205 including a single outlet which generates a single subcutaneous drug depot 1210. In at least one embodiment, the injection means 1215 includes a plurality of pores and/or micro-needles which generate a plurality of depots 1220 (transcutaneously and/or subcutaneously). In at least one embodiment, a combination thereof 1225 is implemented (1228/1210 and 1205/1227).

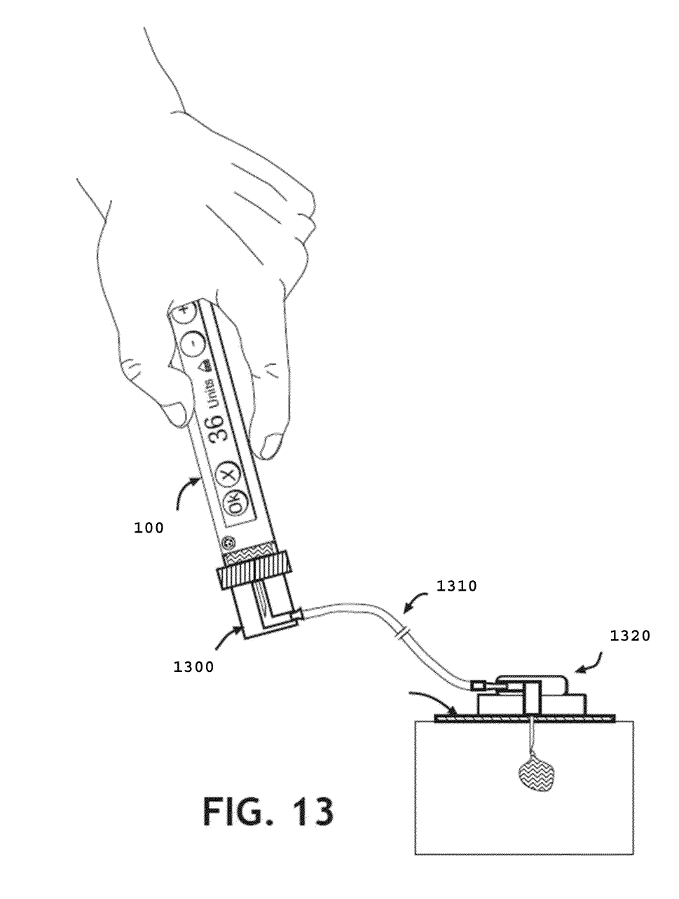

FIG. 13 shows an exemplary injection device coupled to an infusion set (either directly of via an adapter). The insulin injection device 100 receives an adapter 1300 connected via a tubing 1310 to an infusion set 1320. This may allow the device to be used as a temporary replacement for an insulin pump, if, for example, the batteries or reservoir of their insulin pump are exhausted.

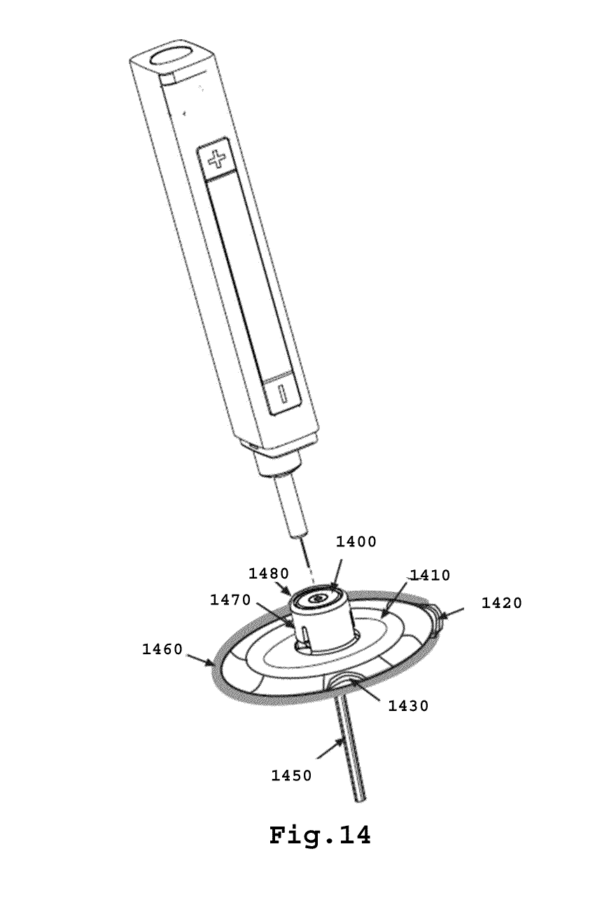

FIG. 14 shows the insulin injection device coupled directly with an infusion port 1400 (disconnection means 1420 and 1430, inlet 1400, needle 1450, adhesive layer 1460, plastic cover 1410).

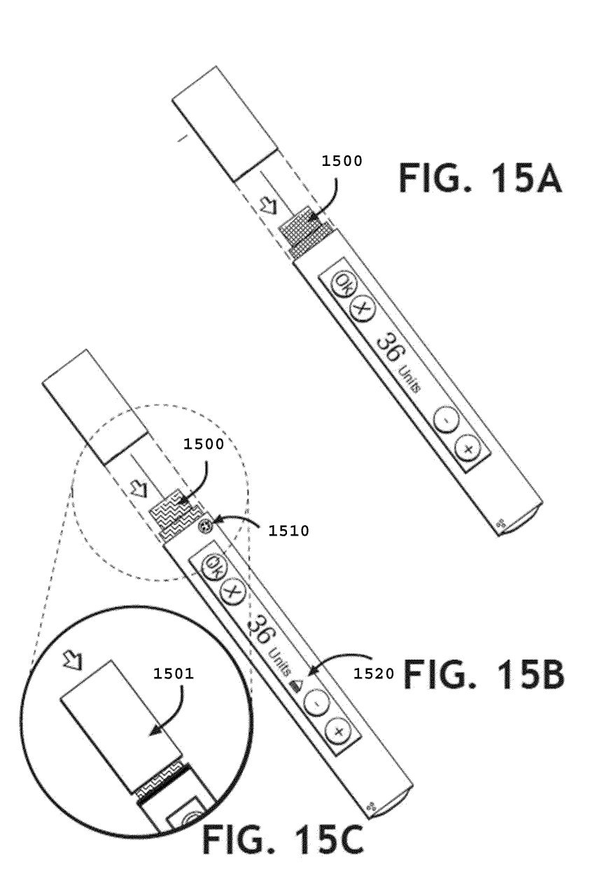

FIGS. 15A to 15C show an exemplary insulin injection device configured to enable the user to observe the cartridge and to have a visual indication with regard to the amount retained within the cartridge. This may be carried out via a transparent portion 1500 comprised in the housing of the injection device or via a gap (e.g., slot, slit) between the injection cap (or cover) and body portion, i.e., the housing.

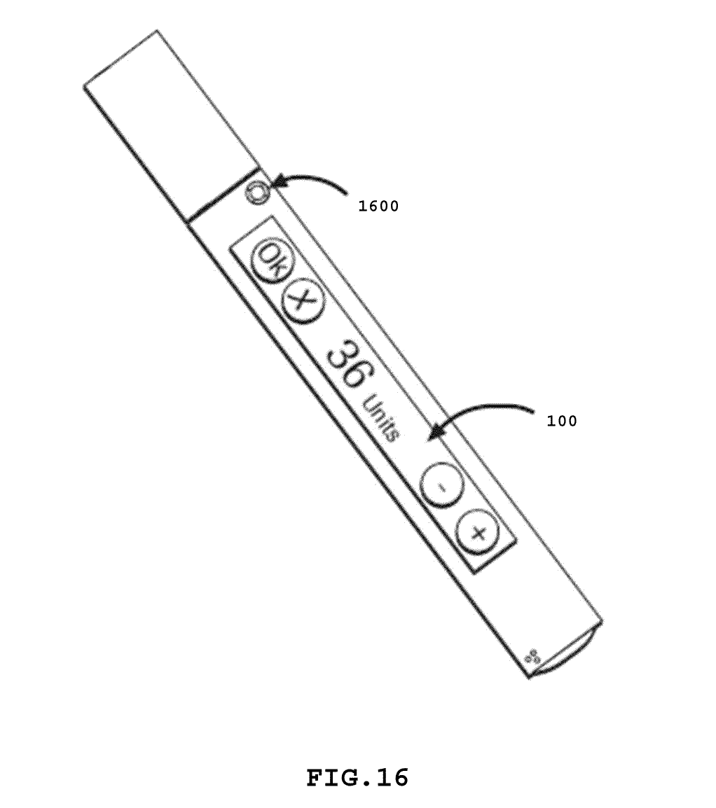

FIG. 16 shows an indicator 1600 notifying the patient whether the cap is properly connected with the housing or not. Such an indication may be configured as a light-emitting diode (LED) operating in conjunction with an electronic sensor which is enabled to determine whether the cap is connected (properly) or disconnected to/from the housing.

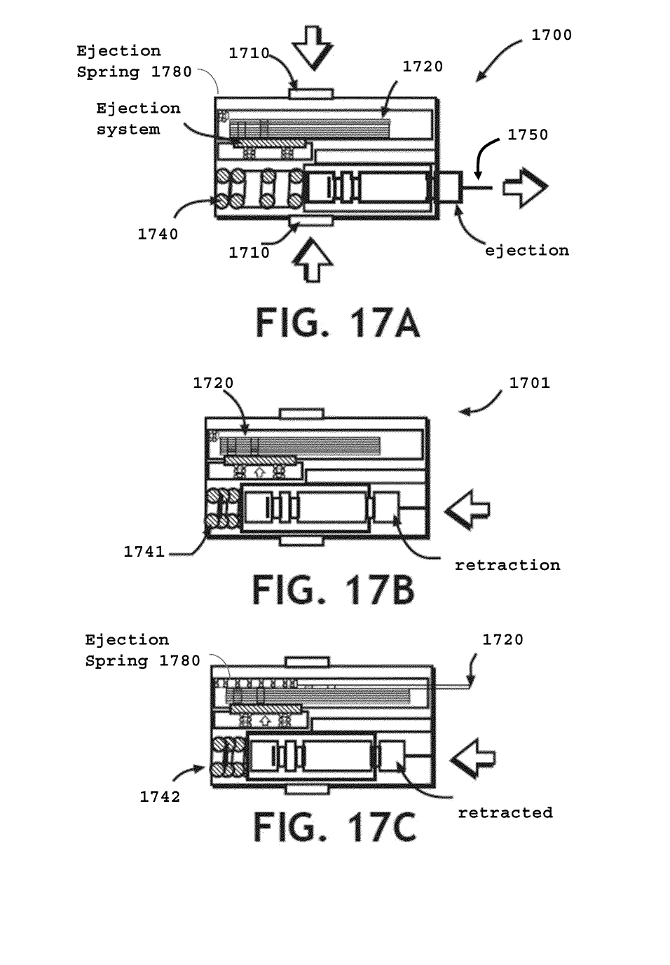

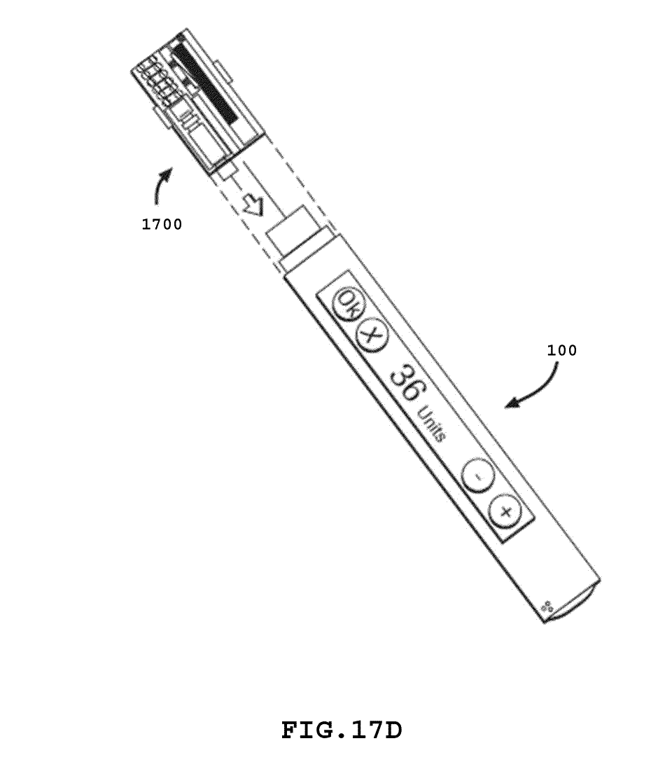

FIGS. 17A to 17C illustrate at least one embodiment of an injection device including a cap for at least protecting the injection needle. The cap is further configured to accommodate one or more of a lancing device, BG strips, injection needle, and other elements or devices to be used by a diabetic patient. In an exemplary embodiment, the user presses the button 1710 and this triggers the release of the needle 1750 (FIG. 17A) by the release of the spring 1740. In FIG. 17B the needle is then retracted and this energizes the spring again 1741 while a test strip is partly ejected (FIG. 17B) due to the release of a previously energized strip test ejection spring 1780.

FIG. 17D shows an embodiment of an insulin injection device 100 provided with an exemplary cap 1700. Appropriate holes or spaces are placed at the distal end of the insulin injection device and in the cap in order to accommodate both the needle of the insulin injection device and the lancing needle of the cap.

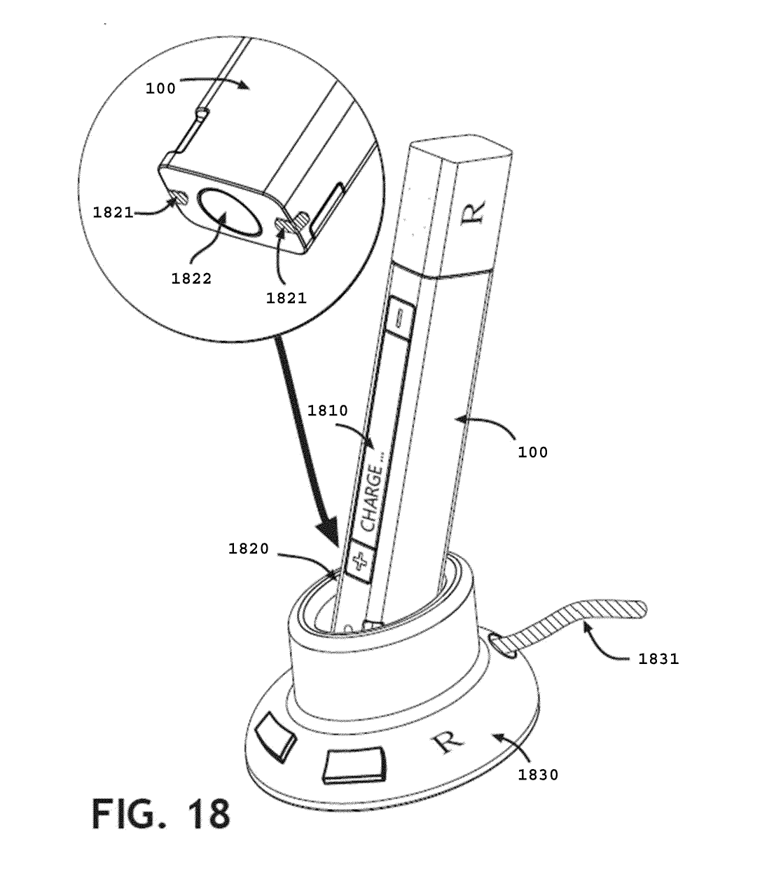

FIG. 18 illustrates an exemplary base station 1830 for receiving an embodiment of insulin injection device 100. In one embodiment, the battery of the pen is rechargeable. When the insulin pen is inserted in the base station 1830 by the opening 1820, the battery is recharged by contact with the electrical contacts 1821. A data transfer can occur by the same electrical components 1821 (all via the communication port 1822). The insulin pen may display the status of the transfer via a display 1810. The base station thus performs a dual function: energy and data transfer. Optionally, the base station can be adapted for automatic refueling of insulin (in this case the entry 1822 enables the refilling). The cable 1831, according to various embodiments, can transmit electricity or insulin or data and or a combination thereof (combinations of tubes and parts). The charging connection may be configured as a wired connection (e.g., USB cable) or a wirelessly connection (e.g., induced charging).

In at least one embodiment, the base station 1830 is connected to the network. If the insulin pen is not provided with communication means, the intermittent connection to the base station enables a corresponding intermittent connection to the network (such as are discussed in particular in FIG. 6)

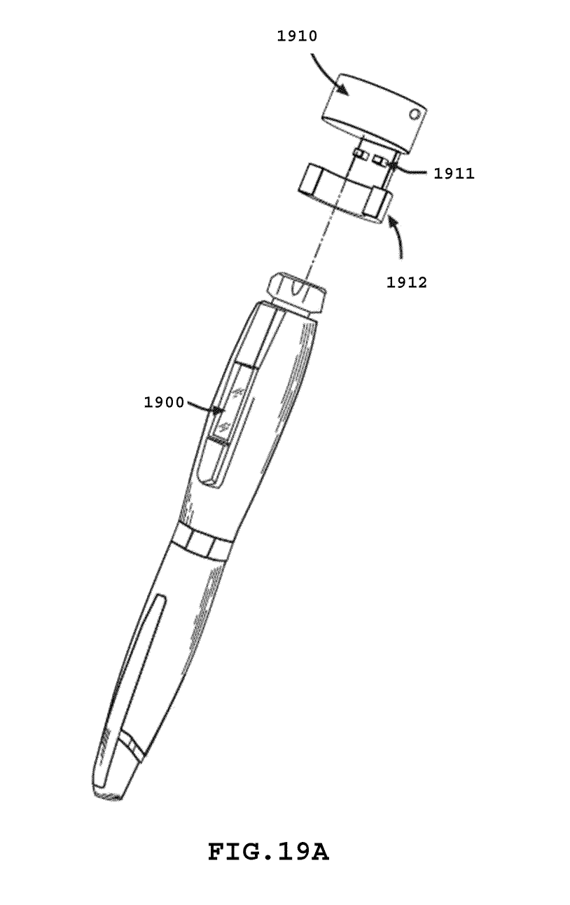

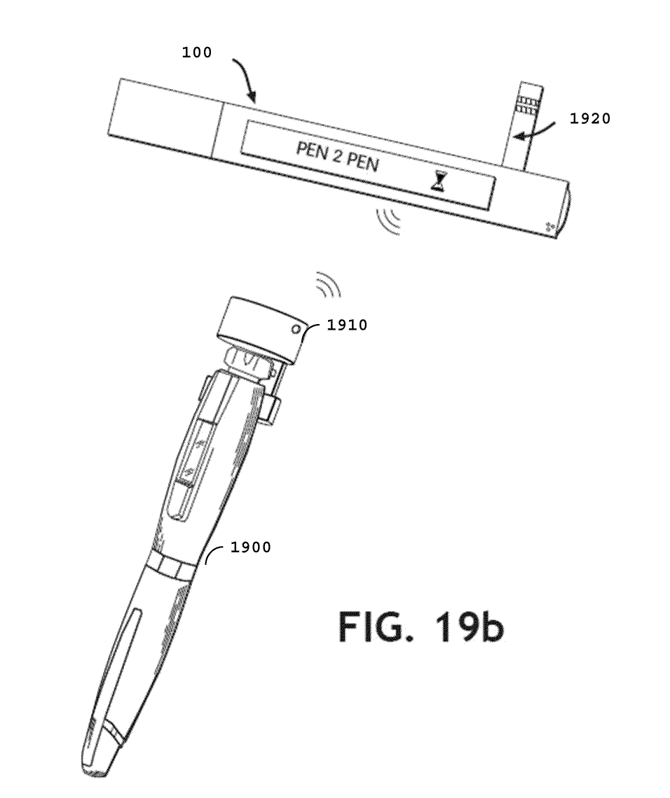

FIG. 19A shows a special communication adapter 1910 for enhancing a standard insulin pen 1900. The adapter acts as a transmitting device which enables to read data from a conventional injection device. The adapter comprises a sensor 1911 and a communication means 1912. The sensor assesses the type and quantity of insulin in the conventional pen and following the results or transmitted via communication means.

FIG. 19B shows the cooperation of a first injection device, namely an “intelligent” insulin injection device 100 (according to the present disclosure) with a second injection device, namely a standard or conventional insulin pen 1900 provided with an embodiment of communication adapter 1910.

When coupled to the first “intelligent” injection device, the transmitting device may be capable of monitoring and recording the amount of insulin injected and/or delivered by the second (conventional) injection device. The corresponding data can be stored in the memory of the “intelligent” device (the insulin pen or injection device according to embodiments of the present disclosure). The processing of data is performed by the first injection device.

In at least one embodiment, one pen can contain rapid acting insulin while the other one would contain long acting insulin. The communication of data can enable the optimization of bolus calculations. Further, the record of injections by one pen enables optimized subsequent injections by the other pen.

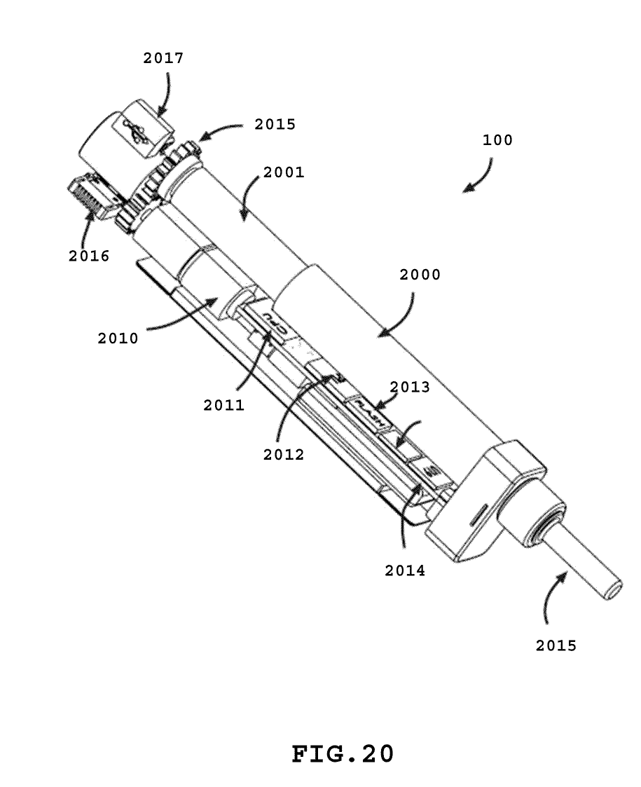

FIG. 20 shows a view of the insulin injection device according to some embodiments of the present disclosure. Exemplary drug injection device 100 comprises a reservoir 2000 (for example containing insulin) with a plunger or piston 2001, activated by gears 2015 connected to a motor 2010. The exemplary drug injection device comprises an analyte meter 2016, for example a blood glucose meter. The insulin injection device further comprises a USB port 2017, a CPU 2011, a memory 2013, a communication unit 2014, a controller or bolus calculator 2012, and a needle 2015 (retractable or not, covered or not). Many different configurations of the drug injection device are possible. For example, the CPU or computing means, the memory means, the controller means can be remotely accessed via a network, instead of presenting local and physical implementation.

Further options and details of the present embodiments may include: a telescopic screw mechanism, USB connectors and isolators, a Li-Ion Polymer Battery, bolus button switch, BGM Connector, encoder, buzzer, cartridge locking mechanism, cartridge identification.

For fluid delivery or drive mechanisms, the further embodiments may include:
Hydraulic Pump (the hydraulic pressure pushes the cartridge's plunger);
Ball Chain (the lifter Motor lifts one ball every time, a special fork takes each time one ball upwards);
Micro Piston Cyclic Pump (one-way valves keeps the insulin going only to the outer needle, requires disposable parts);
Internal “cart” (direct rolling of wheels on the inner part of the cartridge);
Drums Propeller (3 angled rollers move in a thread-like spiral inside the cartridge);
Telescopic “antenna” (a cable pushes the plunger, a telescopic “antenna” supports the cable);
Magnetic Coil (a magnet pushes the plunger, magnetic field comes from a coil outside the cartridge);
Popping Nut (after half travel, the motor goes back, then the outer nut part stays in place, while the inner part extends); and
Telescopic Screw/Nut.

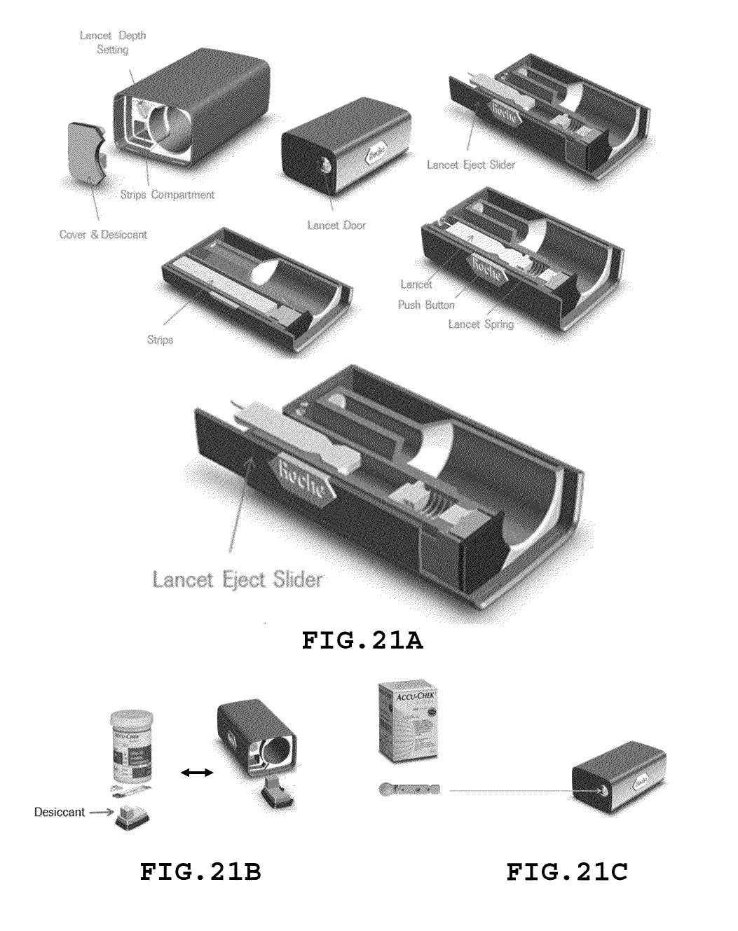

FIGS. 21A to 21C show embodiments of the cap comprising lancing elements and test strips, as well as a strip compartment cover with desiccant.

As shown in FIG. 21A, the cap comprises the lancing elements arranged in a compact form. This exemplary form comprises: a lancet depth setting mechanism (in this case a set screw) that permits a user to adjust the depth of lancet penetration to personal preferences and/or needs, a strip compartment for holding measurement strips such as blood glucose measurement strips, a lancet door (for insertion of disposable lancets), a lancet eject slider (actuated by the user) that can also function as a drive mechanism tensioner, a lancet (disposable or reusable in some embodiments or with a plurality of lancets), a push button (pressed by the user), a lancet spring (one or more for automatic insertion and retraction of lancets), (disposable) strips, a cover including a desiccant compartment for holding a desiccant (the cover can be disposable and/or the desiccant can be renewable such that it is changed periodically to keep the humidity within the strip compartment low; for example, a new cover can be provided to a user in a container along with a certain number of measurement strips), one or more springs (for ejection of strips) In at least one embodiment, the slider, when moved, energizes a spring. The user can adjust the depth setting. When the push button is pressed, the spring is released and the lancet is ejected to the preset depth.

According at least one embodiment, a container will contain test strips and one or more strip compartment covers including a desiccant within or one or more desiccant modules that can be replaced in the cap. The user will thus be able to refill the cap of his/her insulin pen with new disposable test strips and one or more renewed desiccant caps. One of the purposes for this arrangement is to get a subset of the previously available disposable elements (preserving and optimizing portability) and maintaining the test strips in an environment that helps ensure their accuracy and precision over repeated measurements.

As shown in FIG. 21B, the cap is provided with a compartment for strips. The compartment comprises an opening (or a window or an access or an entry or a hole). The opening (or equivalent) is covered by an element (piece of plastic, of metal, of wood, or resin, of polymer) covered by desiccant. As shown in the figure, the element covered by desiccant fits into the opening of the compartment. This arrangement helps makes sure that test strips are maintained in a low humidity environment. A desiccant is a hygroscopic substance that induces or sustains a state of dryness (desiccation) in its local vicinity in a moderately well-sealed container. Desiccants are often solids, and work through absorption or adsorption of water, or a combination of the two. Other desiccant may work through other principles, such as chemical bonding of water molecules. Desiccants remove excessive humidity that would normally degrade or even destroy products sensitive to moisture. Some commonly used desiccants that may be used include: silica gel, activated charcoal, calcium sulfate, calcium chloride, montmorillonite clay, and molecular sieves. Rice or salt are common “low-tech” alternatives.

As shown in FIG. 21C, the cap can also house pre-packaged lancets, of common or proprietary format.

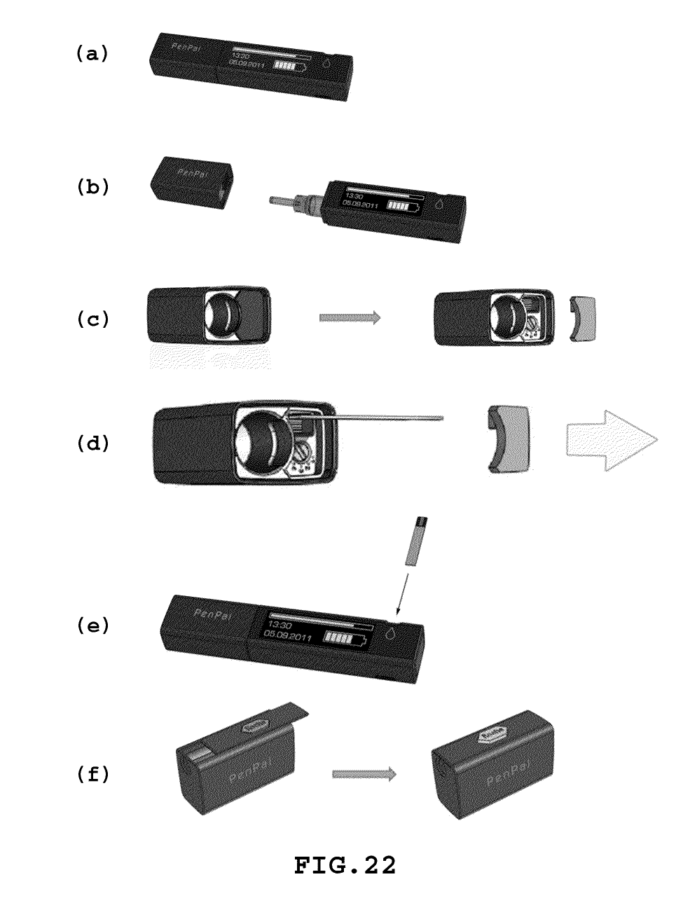

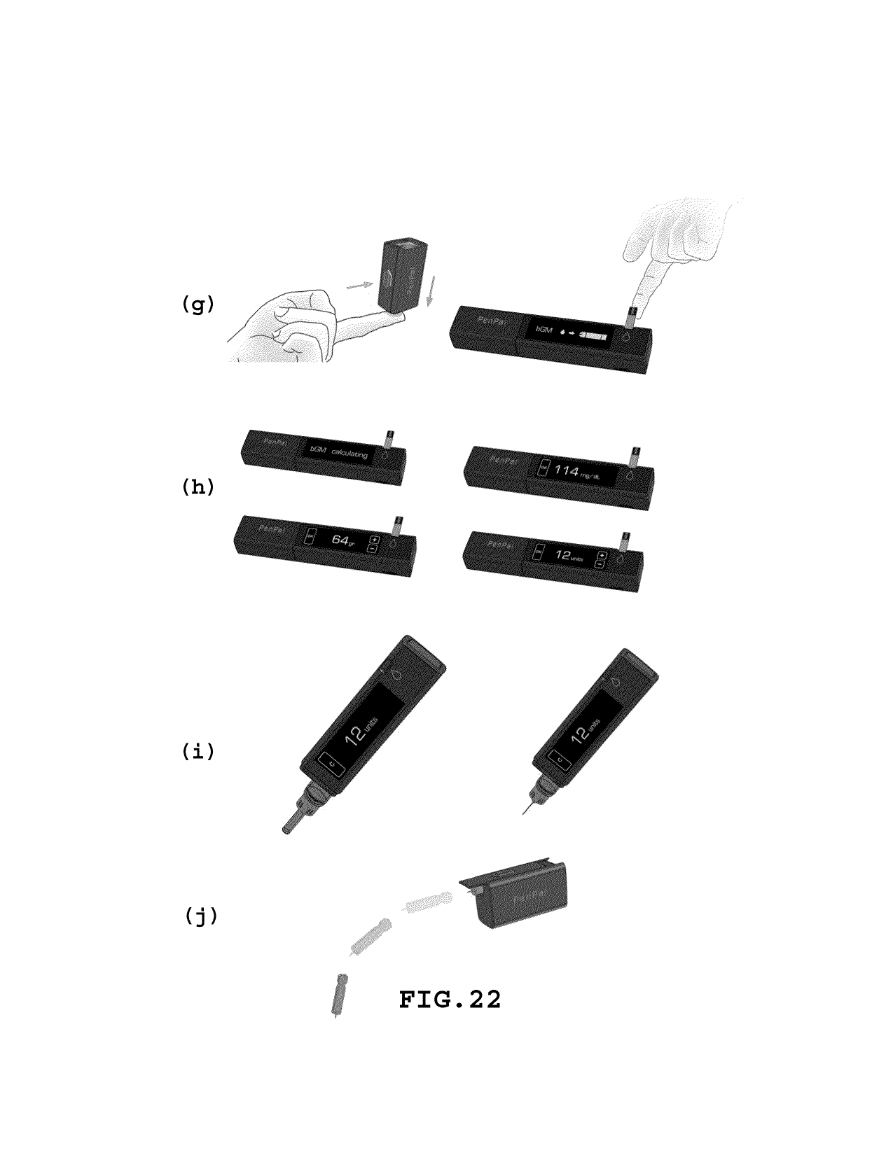

FIG. 22 shows steps of an example of the use of an embodiment of an injection device with the cap comprising lancing elements and test strips. At step (b) the cap is removed (and the injection device can be powered up if a sensor or connector is present to initiate the start-up sequence when the cap is removed, for example, the user interface can switch on, etc., thereby helping to reduce power consumption while the cap is in place but making it unnecessary for the user to do something other than removing the cap to turn the device on). At step (c) the user removes the element (which optionally contains a desiccant as described above) covering the strip compartment. If not done previously as a default during manufacture or previously by the user, the user can access the lancet depth adjustment mechanism once the covering element is removed. The user then extracts manually or automatically a test strip out of the compartment (step (d)). At step (e), the user inserts a test strip into the injection device (in the blood glucose meter for example) At step (f), the user slides the lancet eject slider and this energizes the spring contained in the cap. At step (g) the user pricks his/her finger and immediately after brings the blood in contact with the test strip previously inserted in the blood glucose meter. At step (h) the BGM calculates and then displays the BG value, the user enters a carbohydrate value corresponding to the food intake (consumed or planned in the near future) and the BG displays the recommended bolus. At step (i), the needle is uncovered and the bolus is injected. At step j) the disposable lancing element is removed (and replaced).

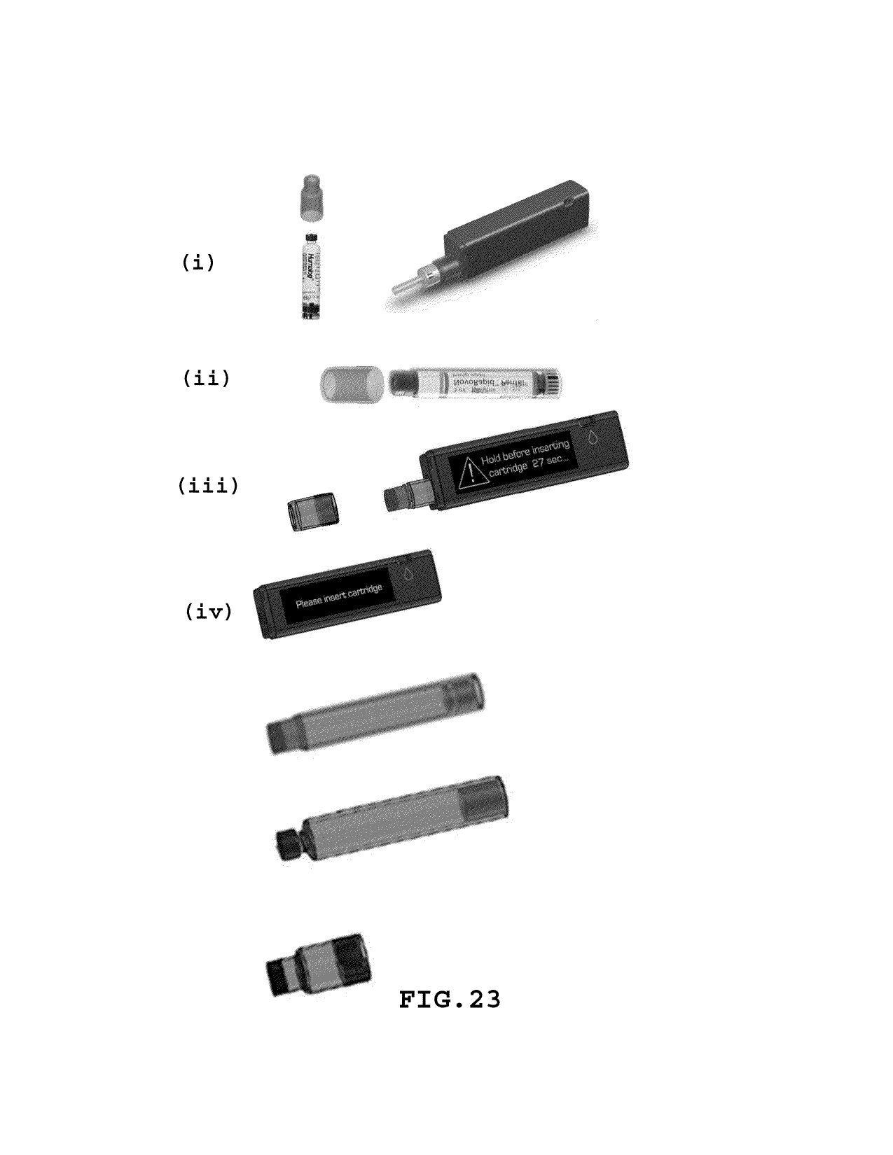

FIG. 23 shows various exemplary adaptors to accommodate different insulin cartridges. i) shows an adapter for a first format ii) shows an adapter for a second format iii) shows the replacement of a cartridge and iv) shows different cartridges format.

In one particular embodiment (not shown), the injection device presents a pen shape, comprises a BGM, a touch sensitive screen; a physical bolus button, a motor, a bolus calculator and a single housing (not including the cover). A scenario of use of an exemplary injection device of the present disclosure is the following: BG level is sensed via BGM (+strip). The result is presented on the Screen. Further, the bolus is calculated based on BG reading and other parameters (e.g., ITC, BOB) are stored in memory. A recommended bolus dose is presented on screen. The dose can be adjusted via touch sensitive screen. Initiation of delivery is carried out following activation of physical bolus button. Delivery of insulin is made possible by the motor-based pumping mechanism. Delivery characteristics are stored in memory. All steps are done within a single housing. As further options: a) BGM is non-invasive (e.g. optical-based) and does not include a strip. Pricking is not required; b) Physical Bolus Button is configured as by voice commands, eye movement or gesture commands; c) Bolus Calculation is based on other physiological parameters (e.g., heart rate, ventilation, blood pressure) which are monitored in real-time; and d) touch-sensitive screen presents information in Braille for visual impaired patients. Optionally, there may be no need for a screen, such as if information is projected on the retina of the user.

Among advantages and effects of embodiments of the present disclosure, the advantages/effects may include: the pen having a memory for better glycemic control because more accurate values are used for calculations; ease of use (user carries a single compact device); accurate delivery (motor-based) better glycemic control; the touch screen enables compact I/O in one device; the physical button for bolus is provided as a safety ; the device is a self-contained compact device—auxiliary components (e.g., remote control, computer, stand-alone glucometer) are not required; the wired communication between delivery and sensing functions reduces the risk of loss of data; the integration of functions increases the robustness of the device and of the data management. In other words, there is described an integrated system for diabetes (bolus) management; or a clinical integrated I/O system (input is BG level, output is delivery of insulin); or an intelligent bolus doser; or a digitalized controlled insulin pen.

According to another embodiment of the present disclosure, the pen is provided with reminder, alert and alarm means. An exemplary scenario is a “duo” bolus pattern, where an initial dose is injected and after a predefined time a reminder prompts for a second bolus dose. Another scenario is a “degraded basal”; the pen is used as a MDI-like device (Multiple Daily Injection) and serves as “backup” for the basal injection (for example one prick per hour may be acceptable in absence of a patch or an insulin pump; alternatively, the user may set the compromise he wants, each 30 min for a degraded bolus for example). A predefined time may be setup, either (a) fixed (b) variable (b1) defined by a logic in the cloud or (b2) defined by a logic implemented in the local networked devices. The context of the user may further be taken into account (the accelerometer can postpone the second bolus by maximum 15 minutes if in a night club for example). Advantages and effects relate to a better system regulation, a better glycemic control. In particular, the first injection may condition the start of the measurement of time and no injection may be possible before the time is elapsed (integration/combination). In other words, the injection device may be “intelligent.” Therefore, embodiments of the present disclosure may be enabling for a semi-automated structured testing.

In yet another embodiment (not illustrated), the pen or injection device comprises communications means and alert/alarm/reminder means. This connection to the network enables alerts to be sent to parents via SMS for example. The connection (even intermittent) enables synchronization with cloud hosted calendars for alerts, alerts and reminders. For example, if and when the USB pen is connected to the network the level of insulin can trigger the purchase of additional cartridges. A real time monitoring of insulin consumption is enabled for insulin manufacturers. Connectivity means retroaction or feedback loops.

Embodiments of the present disclosure also encompass the following alternatives:

An injection device for delivering a fluid, the injection device comprising:
a housing;
a reservoir;
a drive means for expelling the liquid from the reservoir;
a processor for controlling the drive means;
an injection means fluidically coupled to the reservoir and protruding from the housing;
a user interface with data input and data output means; and

characterized in that the injection device comprises a blood glucose meter.

In at least one embodiment, the device may further comprise communications means adapted to transmit blood glucose values measured by the blood glucose meter to a networked medical device, such as an insulin pump or a continuous monitoring device, or to a device such as a personal computer, a tablet, a mobile phone, or a television.

There is disclosed an embodiment of the injection device for delivering a drug, the injection device comprising:
a housing;
an emplacement for a drug cartridge;
a drive means for expelling the liquid from the reservoir;
a processor for controlling the drive means;
an injection means fluidically coupled to the reservoir and protruding from the housing;
a user interface with data input and data output means; and

characterized in that the injection device comprises a drug cartridge identification mechanism, the identification mechanism enabling the retrieval of data such as type of insulin, expiry date, manufacturer, or batch or lot.

In at least one embodiment, the identification mechanism includes elements such as bar code, color detection, RFID, hologram, OCR or a combination thereof.

There is disclosed an authentication mechanism resulting in the acceptance or the rejection of a drug cartridge.

In an exemplary communications means, the amount of remaining drug in the cartridge being communicable via a network.

In at least one embodiment of an injection device for delivering a fluid of the present disclosure, the injection device comprises:
a housing;
a reservoir;
a drive means for expelling the liquid from the reservoir;
a processor for controlling the drive means;
an injection means fluidically coupled to the reservoir and protruding from the housing;
a user interface with data input and data output means; and

characterized in that the injection device comprises a cap for covering the injection means and the cap is configured to house lancing elements such as a lancet and a test strip.

In at least one embodiment, the cap further houses one or more springs adapted to expel or retract the lancet.

In at least one embodiment of an injection device for delivering a fluid of the present disclosure, the injection device comprises:
a housing;
a reservoir;
a drive means for expelling the liquid from the reservoir;
a processor for controlling the drive means;
an injection means fluidically coupled to the reservoir and protruding from the housing;
a user interface with data input and data output means; and

characterized in that the user interface comprises a microphone and voice recognition means adapted to perform tasks such as receiving voice commands, recording comments or assessing audio ambience.

Further, in at least one embodiment, an audio speaker is adapted to provide audio feedbacks, such as confirmations, to the user.

Exemplary voice synthesis means may also be adapted to restitute data such as a blood glucose value or a confirmation of an injection.

In at least one embodiment of the present disclosure, the disclosure also encompasses the following exemplary devices:
an injection and measurement device wherein the lancing means and/or the injection means can be actuated by voice commands such as “test” or “prick” or “inject” received by the microphone;
an injection device wherein the lancing means or the injection means can be actuated by voice commands received by a microphone implemented on one of a networked device, such as a mobile phone, in communication with the injection device;
an injection and measurement device which, when triggered (such as by voice command, button pressed, or gesture), pricks the skin of a patient, withdraws a blood sample, analyses the blood, establishes a drug remediation and immediately after (i.e. without a confirmation by the user but according to predefined agreed injections schemes for example comprising thresholds and maximal amounts of drugs over certain periods of time) injects the drug remediation, using the same withdrawal needle or a second and distinct injection means;
an injection device provided with network connectivity and which continuously monitors available and connected medical devices in the proximity of the patient, establishes a strategy of injection, and upon opportunity injects one or more drugs (injection of a basal dose while a bolus is injected);
an injection device provided with communications means which communicates data, comprising parameters such as the remaining volume of insulin in one or more cartridges, or the number of remaining test strips, to one or more networked devices, which devices in turn can order cartridges or test strips for replacement or to perform other related tasks;
an injection device provided with a plurality of cartridges or reservoirs and with computing means, local or remotely accessed, adapted to manage multiple injection schemes and associated dosage administration;
an injection device compatible with a standard needle or cannula or a specific cannula such as a sprinkler cannula, the mode of injections being controllable by the user interface or by voice commands;
an injection and/or measurement device provided with communications means which transmits measured diagnostics values to the networked devices present nearby and wherein the devices display one or more of the values;
an injection and/or measurement device provided with communications means which receives data from other networked devices present nearby and which takes the data into account for the drug administration and/or display the data on the injection and/or measurement device;
an injection and/or measurement device provided with insertion means for cartridges, such as an opening, window, hole, shield, plug or a combination thereof;
an injection and/or measurement device provided with a cap covering the injection means and a sensor determining whether the cap covers the injections means (open position) or not (closed position) and wherein the screen or user interface sleeps or hibernates in open position and lights on or activates when in closed position.

The disclosure also encompasses exemplary embodiments of injection devices, which include:

a) an injection device provided with cartridge recognition means;

b) an injection device provided with a plurality of cartridges and/or reservoirs;

c) an injection device provided with voice command means;

d) an injection device provided with a cap containing lancing elements such as lancets and test strips;

e) a networked injection device provided with communications means;

f) an injection device provided with a slider (physical element or software embodiment);

ab) an injection device provided with cartridge recognition means and a plurality of cartridges and/or reservoirs; wrong or adapted insertions can be detected and associated warnings can be raised; a management of remaining drugs is enabled (for example, rapid acting insulin can be substituted to long acting insulin by adapting the number and/or frequency of injections);

ac) an injection device provided with cartridge recognition means and voice command means; voice commands can activate or confirm the loading of the identified insulin;

ad) an injection device provided with cartridge recognition means; with a cap containing lancing elements such as lancets and test strips; the number and type of the lancets and/or test strips can be correlated to the type of drug contained in the identified cartridge;

ae) an injection device provided with cartridge recognition and with communications means; the amount of remaining insulin can be monitored and reported;

af) an injection device provided with cartridge recognition means and with a slider (physical element or software embodiment); the slider, in one position, on demand or a certain time intervals, can represent the amount of remaining insulin (no need for a real visual control with a transparent housing for example); the present control is a virtual one, a representation enabled by the position of the cursor;

bc) an injection device provided with a plurality of cartridges and/or reservoirs and with voice command means; for example, the user can choose by voice command whether he wants to inject rapid or long insulin or both;

bd) an injection device provided with a plurality of cartridges and/or reservoirs and with a cap containing lancing elements such as lancets and test strips; the number and type of the lancets and/or test strips can be correlated to the number and type of drugs contained in the plurality of cartridges and/or reservoirs;

be) an injection device provided with a plurality of cartridges and/or reservoirs and with communications means; the management of cartridges can be controlled by or via the network (order, reimbursement, expiry date management, etc.);

cd) an injection device provided with voice command means and with a cap containing lancing elements such as lancets and test strips; the prick can be actuated by a voice command for example;

ce) an injection device provided with voice command means and with communications means; for example, a voice command can be transmitted over the network and partly executed on or by the injection device (injection, calibration, etc.) and partly on or by the network (record of the audio comment, logbook, command to another device, etc.);

cf) an injection device provided with voice command means and with a slider (physical element or software embodiment of the user interface); for example, after a voice command is received, the slider moves, the user looks at the slider to confirm the amount of insulin to be injected. A bright and big bar as a slider is easy to see and valuable for visually impaired;

de) an injection device provided with a cap containing lancing elements such as lancets and test strips and communications means; the number of remaining lancing elements can be monitored and reported;

abc) an injection device provided with cartridge recognition means, a plurality of cartridges and/or reservoirs and voice command means; wrong or adapted insertions can be detected and associated warnings can be raised; a management of remaining drugs is enabled by voice command (for example, rapid acting insulin can be substituted to long acting insulin by adapting the number and/or frequency of injections);

abe) an injection device provided with cartridge recognition means; with a plurality of cartridges and/or reservoirs; with communications means; the management and monitoring of cartridges is enabled, as well as inappropriate insertions for example;

bcd) an injection device provided with a plurality of cartridges and/or reservoirs; with voice command means; with a cap containing lancing elements such as lancets and test strips; voice commands can control the lancing process and/or the injection process.

Exemplary embodiments and aspects of the embodiments may also include:
a cartridge stability aspect wherein the pen is provided with a reading unit to read out information about the stability of a fluid within a cartridge (it comprises an optical unit which measures the transparency of the fluid);
a cartridge lock wherein once inserted the removal of the cartridge is detected;
a test strip vial with a lid and test strips and desiccant packages which can be removed to be inserted into the chamber of the above mentioned cap;
a lancet device with a hole through which a lancet can be exchanged, whereby the size of the hole is automatically reduced by a closure during cocking;
a lancet device with a slider for cocking the drive unit if the slide is moved in the first direction and ejecting the lancet by moving the slider in another direction;
a pen with a cap and a lancet device integrated into the cap whereby the lancet can only be cocked if the cap is removed from the pen;
a cap for protecting an injection needle having two further separated compartments for inserting a lancet and test strip whereby the two compartments can be closed and opened independently from each other;
a device with a measuring unit for measuring an analyte having a processor for computing a value of the measured analyte and a display for showing the value whereby based on the measured value a bolus is calculated and shown on the display and a user interface allowing the user to change or confirm the shown bolus and an adjusting unit for automatically adjusting a fluid dispensing system in accordance to the changed or confirmed value.

In at least one embodiment, the drug injection device is a connected device, but in another embodiment it may operate as a stand-alone device (online, off-line and intermediate modes). The drug injection device interacts with other networked medical devices, such as pens or insulin pumps or continuous monitoring sensors. It can also interact with so-called Body Area Network (BAN) sensors or Body Sensor Network (BSN). Body sensors comprise wearable and/or implementable and/or wireless (bio) sensors adapted to assess the physiological state of the user or patient. These sensors can monitor the physiological state continuously or not (on demand or upon action of the user). For example, the following parameters can be monitored: blood glucose, heart rate, body temperature, ECG, EEG, EMG, sweat, blood cholesterol, blood alcohol, coagulation, and estimation of physical exercise or carbs consumption (such as evaluated by accelerometers or pedometers for example). The drug pen or injection device is thus part of an integrated medical system. This means in particular that opportunistic approaches are possible: for example, when visiting a diabetic friend, the bolus or basal doses can be administrated with increased flexibility by using devices of third parties, drug administration doses can be fractioned, etc. The drug administration can be distributed over several—and available—medical devices (the presence of such devices can be continuously monitored in the proximity of the patient).

As examples of the value emerging from the cooperation of networked medical devices, the following examples (situations and actions) are provided: a) according to the user profile, for example a child, do a measure at 11 am (highest risk of hypo); b) with an accelerometer, detect excessive shocks; c) with a thermometer, detect excessive or fluctuating temperatures; combined with a degasing model, prompt the user to check tubing for air bubbles; d) if the reservoir becomes too low, combined with an awareness of calendar/schedule, the emergency of refilling triggers an alert or not (a reservoir low at 10 am after breakfast bolus is not as important as a low reservoir at 13 pm while lunch occurs); and e) with a GPS, assess the life situation (for example travelling, known by GPS data) and take further appropriate actions (in car, in plane, working, sleeping, etc.). The cooperation of networked medical devices leads to a better disease management, by taking into account more exogenous parameters (anticipation of life events).

Further disclosed is a exemplary drug injection device comprising a drug delivery module and an analyte measurement module, such as a blood glucose meter. The device can be provided with I/O means and can be further adapted to communicate with other networked medical or IT devices. Various options are disclosed, which include: different energy sources, a code reader adapted to identify a previously coded drug cartridge during or after insertion, a plurality of different drug cartridges, a sprinkler needle to optimize injections, an adaptor for connection to an infusion set or to a micro pump, a cap covering the needle and containing lancing elements as well as blood test strips, an accessory base station for energy transfer and/or data exchange. In at least one embodiment, the sprinkler needle optimizes the insulin depots during injection. Associated systems and methods are disclosed.

While various embodiments of devices, systems, and methods of administering a drug to a patient have been described in considerable detail herein, the embodiments are merely offered by way of non-limiting examples of the disclosure described herein. It will therefore be understood that various changes and modifications may be made, and equivalents may be substituted for elements thereof, without departing from the scope of the disclosure. Indeed, this disclosure is not intended to be exhaustive or to limit the scope of the disclosure.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. Other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. An injection device for delivering a fluid, the injection device comprising:
a support for receiving a reservoir or a cartridge;
a drive means for expelling the fluid from the reservoir or cartridge; and
an injection means fluidically coupled to the reservoir or cartridge;
wherein the injection device comprises an analyte measurement module.

2. The injection device of claim 1, wherein the delivery of the fluid corresponds to a prick, or is configured to oscillate back and forth along a longitudinal axis as the liquid is dispensed from the fluid injection means.

3. The injection device of claim 1, wherein the analyte measurement module is part of a housing of the injection device.

4. The injection device of claim 1, wherein the analyte measurement module is a blood glucose meter and the fluid is insulin.

5. The injection device of claim 1, further comprising a user interface including data inputting and outputting means.

6. The injection device of claim 1, further comprising communication means, adapted to communicate with other networked devices.

7. The injection device of claim 1, further comprising an energy source selected from the group consisting of a dynamo, a spring, a rechargeable battery, a solar cell, or a combination thereof.

8. The injection device of claim 1, further comprising a code reader adapted to identify a previously coded drug cartridge.

9. The injection device of claim 1, further comprising a plurality of drug cartridges and/or drug reservoirs.

10. The injection device of claim 1, further comprising an injection needle operably coupled to the injection means.

11. The injection device of claim 10, wherein the needle is a sprinkler needle comprising a plurality of holes.

12. The injection device of claim 10, further comprising an adapter to the needle, the adapter being connectable via a tubing to an infusion set or a patch-pump inlet.

13. The injection device of claim 10, further comprising a cap covering the needle, the cap containing lancing elements and one or more blood test strips compatible with the blood glucose meter.

14. The injection device of claim 1, further adapted to be received in a base station, wherein the base station is suitable for energy transfer and/or data exchange.

15. A medical system comprising:
a pen-shaped medical device comprising:
a support for receiving a reservoir or a cartridge,
a drive means for expelling the fluid from the reservoir or cartridge, and
an injection means fluidically coupled to the reservoir or cartridge,
wherein the injection device comprises an analyte measurement module, and
a computing means adapted to implement one or more functions of a bolus calculator, the implementation of the computing means being local, for example in a processor, or the computing means being accessed remotely via a network.

16. The medical system of claim 15, further comprising a plurality of cooperating pen-shaped medical devices and/or with a standard pen mounted with a communication cap comprising a sensor and communications means.
wherein, each of the plurality of cooperating pen-shaped medical devices comprise:
a support for receiving a reservoir or a cartridge,
a drive means for expelling the fluid from the reservoir or cartridge, and
an injection means fluidically coupled to the reservoir or cartridge,
wherein the injection device comprises an analyte measurement module.
